(19) United States
(12) Patent Application Publication
(10) Pub. No.: US 2023/0381139 A1
(43) Pub. Date: Nov. 30, 2023

(54) METHODS OF TREATING AND/OR PREVENTING ALZHEIMER’S DISEASE WITH R-CARVEDILOL

(71) Applicant: UTI LIMITED PARTNERSHIP, Calgary (CA)

(72) Inventors: Sui Rong Wayne CHEN, Calgary (CA); BACK Thomas, Calgary (CA); Jinjing YAO, Calgary (CA)

(21) Appl. No.: 18/026,397

(22) PCT Filed: Sep. 17, 2021
(86) PCT No.: PCT/CA2021/051303

§ 371 Date: Mar. 15, 2023

Related U.S. Application Data
(60) Provisional application No. 63/079,508, filed on Sep. 17, 2020.

(51) Int. Cl. A61K 31/403; A61P 25/28

(52) U.S. Cl. CPC A61K 31/403; A61P 25/28

(57) ABSTRACT

Methods of treating and/or preventing dysfunctions associated with Alzheimer's Disease such as memory loss, hippocampal long-term potentiation impairment, neuronal hyperactivity, and neuronal cell death using R-carvedilol, a metabolite thereof, and/or a salt thereof. Also described are related uses and pharmaceutical compositions.

This application claims priority from U.S. provisional application 63/079,508, filed Sep. 17, 2020, the entire contents of which are incorporated herein by reference.

### FIELD OF THE INVENTION

The present disclosure relates generally to neurological diseases or disorders, such as Alzheimer's Disease, and methods for treating or preventing them.

### BACKGROUND OF THE INVENTION

Alzheimer's Disease (AD) is the most common form of dementia and afflicts a rapidly growing population globally. Despite a substantial worldwide effort, there is currently no effective treatment or cure for AD. Over the past several decades, AD research has largely targeted the amyloid cascade that is thought to drive AD progression due to deposition of β-amyloid (Aβ) plaques in the brain (Berridge, 2010; Hardy and Selkoe, 2002; Karran et al., 2011). Hence, the dominating therapeutic anti-AD strategy has been reducing Aβ depositions (Demattos et al., 2012; Kennedy et al., 2016; Sevigny et al., 2016) or increasing their clearance. The majority of Aβ-targeted clinical trials to date have been unsuccessful (Chakroborty and Stutzmann, 2014; Honig et al., 2018; Karran et al., 2011). In June 2021, however, the FDA, based on ambiguous clinical trial results, approved Aducanumab through its accelerated approval pathway. Aducanumab is an amyloid beta-directed monoclonal antibody that targets aggregated forms of Amyloid beta (Aβ) found in the brains of people with Alzheimer's Disease to reduce its buildup. The clinical findings for Aβ-targeted therapies highlight the urgent need to develop new non-Aβ-targeted AD therapeutic strategies (Loera-Valencia et al, 2019; Weller and Buson, 2018; Coman and Names, 2017).

Some studies point to neuronal hyperactivity as an early primary neuronal dysfunction in human AD patients as well as animal models of AD (Busche et al., 2012; Busche et al., 2008; Busche and Konnerth, 2015; Dickerson et al., 2005; Keskin et al., 2017; Lerdkrai et al., 2018; Nuriel et al., 2017; O'Brien et al., 2010; Stargardt et al 2015; Zott et al 2019). Other studies suggest that neuronal hyperactivity can be induced in vivo by acute treatment with exogenous soluble Aβ (Busche et al., 2012; Keskin et al., 2017; Zott et al., 2019). It appears that neuronal hyperactivity itself can trigger the release of endogenous soluble Aβ (Cirrito et al., 2005; Kamenetz et al., 2003; Yamamoto et al., 2015). Thus, soluble Aβ not only may make active neurons more active (hyperactive) but may also trigger soluble Aβ release that in turn may further promote hyperactivity. Some authors believe that this vicious cycle drives Aβaccumulation, neuronal hyperactivity, circuit dysfunction, and AD progression (Busche and Konnerth, 2015, 2016; Stargardt et al., 2015; Zott et al., 2019).

Ryanodine receptor 2 (RyR2) is an intracellular Ca²⁺ release channel. RyR2 is predominantly expressed in the heart and brain, especially in the hippocampus and cortex (Berl, 2002; Furuichi et al., 1994; Giannini et al., 1995; Murayama and Ogawa, 1996). RyR2-mediated Ca²⁺ release plays a role in regulating membrane excitability of various cells (Alkon et al., 1998; Bogdanov et al., 2001; Mandikian et al., 2014; Nelson et al., 1995). Enhanced RyR2 function can cause cardiac arrhythmias and sudden death; and has also been implicated in AD pathogenesis (Bruno et al., 2012; Chakroborty et al., 2009; Kelliher et al., 1999; Lacampagne et al., 2017; Oules et al., 2012; Priori and Chen, 2011; SanMartin et al., 2017; Smith et al., 2005). Thus, targeting RyR2 may be a means to control membrane excitability and neuronal hyperactivity. However, given the multiple essential physiological roles of RyR2, dramatically blocking RyR2 function or expression would be detrimental (Ground et al., 2012; Liu et al., 2014; Takeshima et al., 1998). The challenge is how to suppress overactive RyR2 without detrimental impact.

Carvedilol is a non-selective beta- and alpha-adrenergic receptor blocker that has also previously been identified as a small molecule inhibitor of ryanodine receptor (RyR2) (Zhou et al., 2011). Carvedilol reduces store-overload-induced Ca²⁺ release. Animal studies showed that chronic treatment with racemic carvedilol significantly reduced the content of oligomeric Aβ in the brain (Wang et al., 2011). However, a recent pilot clinical trial of carvedilol (racemic mixture at a dose of 25 mg per day) showed no significant improvement in AD (https://www.clinicaltrials.gov/ct2/show/study/NCT01354444).

The disclosure of all publications, patents, and published patent applications referred to herein are hereby incorporated herein by reference in their entirety.

Thus, there remains a need for methods of treating and/or preventing Alzheimer's Disease and dysfunctions associated therewith.

### SUMMARY OF THE INVENTION

The inventors have found that the administration of R-carvedilol was effective to ameliorate neuronal hyperactivity, impairment of hippocampal long-term potentiation, neuronal cell death, memory loss, and learning deficits in two mouse models of Alzheimer's Disease.

Accordingly, a first aspect of the invention provides a method of treating or preventing at least one, two, three, or four of the following in a subject in need thereof (a) memory loss (including rescuing or at least partially restoring memory); (b) long-term potentiation impairment (including preventing and/or mitigating long-term potentiation impairment); (c) neuronal cell death (including reducing the number of neuronal cell deaths); and (d) neuronal hyperactivity, the method comprising, consisting essentially of, or consisting of administering a therapeutically effective amount of R-carvedilol, a metabolite of R-carvedilol, or a salt thereof to the subject.

The method can be for treating or preventing Alzheimer's Disease, which can be preclinical Alzheimer's Disease, Alzheimer's Disease with mild cognitive impairment, or Alzheimer's dementia. For example, the method can be for use in preventing Alzheimer's Dementia in a subject diagnosed (formally or informally) with Alzheimer's Disease. Alternatively, the method can be for preventing Alzheimer's Disease in a subject at risk of developing Alzheimer's Disease.

The method can also be for treating or preventing cognitive decline, including at least partially restoring cognitive function. For example, the method can be for preventing cognitive decline in a subject at risk of developing Alzheimer's Disease.

In accordance with a second aspect of the present invention, there is provided a method of treating Alzheimer's Disease in a subject in need thereof, comprising, consisting essentially of, or consisting of administering a therapeutically effective amount of R-carvedilol, a metabolite of R-carvedilol, or a salt thereof to the subject.

In accordance with a third aspect, the invention provides a pharmaceutical composition for use in a method according to the first aspect, the pharmaceutical composition comprising, consisting essentially of, or consisting of a pharmaceutically active ingredient consisting of R-carvedilol, a metabolite of R-carvedilol, or salt thereof, together with a pharmaceutically acceptable carrier.

In accordance with a fourth aspect, there is provided a use of a pharmaceutically active ingredient comprising, consisting essentially of, or consisting of R-carvedilol, a metabolite of R-carvedilol, or salt thereof in a method according to the first aspect.

A fifth aspect of the invention provides a use of a pharmaceutically active ingredient comprising, consisting essentially of, or consisting of R-carvedilol, a metabolite of R-carvedilol, or salt thereof in the manufacture of a medicament for use in a method according to the first aspect.

These and other aspects of the invention will be described further below.

### BRIEF DESCRIPTION OF THE FIGURES

Embodiments of the present disclosure will now be described, by way of example only, with reference to the attached Figures.

FIG. 1 shows that the RyR2 E4872Q^{+/-} mutation decreases neuronal excitability and upregulates A-type K⁺ current of CA1 neurons. Images of GFP-tagged RyR2 (A, C) and RyR2-WT (GFP-negative) (B, D) mouse brain sections. (E) Traces of action potential (AP) firing. (F) Current thresholds for AP firing in WT (10 mice, 30 neurons), 5×FAD^{+/-} (10 mice, 30 neurons), 5×FAD^{+/-}/EQ^{+/-} (10 mice, 30 neurons) and EQ^{+/-} (10 mice, 30 neurons) CA1 neurons. (G) Current injection-triggered AP firing frequency in WT (10 mice, 30 neurons), 5×FAD^{+/-} (10 mice, 30 neurons), 5×FAD^{+/31} EQ^{+/-} (10 mice, 30 neurons) and EQ^{+/31} (10 mice, 30 neurons) CA1 neurons. (H) Traces of A-type K⁺ current (I_{A}) from 3-4 months old WT, 5×FAD^{+/-}, 5×FAD^{+/-}/EQ^{+/-} and EQ^{+/-} CA1 neurons. (I) Normalized I_{A} traces. (J) Steady-state activation curves of I_{A}. (K) I_{A} amplitude in 3-4 months old WT (10 mice, 30 neurons), 5×FAD^{+/-} (10 mice, 30 neurons), 5×FAD/EQ^{+/-} (10 mice, 30 neurons) and EQ^{+/-} (10 mice, 30 neurons) CA1 neurons. (L) I_{A} inactivation kinetics (Tau) from the same number of neurons as in (K). (M) The midpoints of voltage-dependent activation of I_{A}(V_{A}) for WT (10 mice, 30 neurons), 5×FAD^{+/-} (10 mice, 30 neurons), 5×FAD^{+/-}/EQ^{+/-} (10 mice, 30 neurons) and EQ^{+/-} (10 mice, 30 neurons) CA1 neurons. Current threshold for AP firing (N) and current injection-triggered AP firing frequency (O) without or with the Kv4 channel agonist NS5806 (10 μM) in 5×FAD^{+/31} CA1 neurons (7 mice, 14 neurons). Whole cell I_{A} (P), inactivation time constant Tau (Q) in Kv4.2/KChIP4 transfected HEK293 cells expressing RyR2 WT (n=21), RyR2 E4872Q^{+/-} mutation (n=26). (R) Biotin-labelled surface Kv4.2 in Kv4.2/KChIP4 transfected HEK293 cells expressing RyR2 WT or RyR2 E4872Q^{+/-} mutation. Scale bar: 10 μm. Data shown are the median and range (Kruskal-Wallis test with Dunn-Bonferroni post hoc test, Wilcoxon matched-pairs signed rank test and Mann-Whitney U test; *P<0.5, **P<0.01 vs WT, ^{##}P<0.01, 5×FAD^{+/-}/EQ^{+/-} vs 5×FAD^{+/-}, NS, not significant).

FIG. 2 shows the effect of RyR2-E4872Q^{+/-} on the afterhyperpolarization current (I_{AHP}), the A-type K³⁰ current (I_{A}), and surface expression of Kv4.2, and the effect of pharmacological manipulation of RyR2 on I_{A}. (A) Representative traces of whole-cell voltage-clamp recording of afterhyperpolarization current (I_{AHP}) from 3-4 months old WT, 5×FAD^{+/-}, 5×FAD^{+/-}/EQ^{+/-} and EQ^{+/-} CA1 pyramidal neurons. I_{AHP} was evoked by a 100 ms depolarizing voltage step to +60 mV from a holding potential of −85 mV, The amplitude of the medium (I_{mAHP}) was measured as peak current and slow (I_{sAHP}) amplitude was measured 1 s after the end of the depolarizing pulse, respectively. Mean amplitude of I_{mAHP} (B) and I_{sAHP} (C) recorded from WT (10 mice, 19 neurons), 5×FAD^{+/-} (10 mice, 22 neurons), 5×FAD^{+/-}/EQ^{+/-} (10 mice, 22 neurons) and EQ^{+/-} (10 mice, 19 neurons) CA1 neurons. (D) Representative traces of membrane potential recordings showing action potential (AP) firing after injecting 150 pA current in 3-4 months old 5×FAD^{+/-} mouse CA1 neurons before and after 10 μM NS5806. (E) Steady-state inactivation curves of whole cell A-type K⁺ currents (I_{A}) obtained from 3-4 months old WT, 5×FAD^{+/-}, 5×FAD^{+/-}/EQ^{+/-}, and EQ^{+/-} mouse CA1 neurons. Data points were fitted using the Boltzmann function. (F) Comparison of V_{H} recorded from WT (10 mice, 30 neurons), 5×FAD^{+/-} (10 mice, 30 neurons), 5×FAD^{+/-}/EQ^{+/-} (10 mice, 30 neurons) and EQ^{+/-} (10 mice, 30 neurons) CA1 neurons. Comparison of midpoints of I_{A} voltage dependent activation (V_{A}) (G) and midpoints of I_{A} voltage dependent inactivation (V_{H}) (H) recorded in Kv4.21KChIP4 transfected HEK293 stable cell lines expressing RyR2 WT (n=21), RyR2 E4872Q^{+/-} mutation (n=26). Comparison of whole cell I_{A} (I), inactivation time constant Tau (J), V_{A} (K) and V_{H} (L) recorded in Kv4.2 (alone) transfected HEK293 stable cell lines expressing RyR2 WT (n=21) or RyR2 E4872Q^{+/-} mutation (n=20). (M) Immunoblotting with anti-Kv4.2 antibody (abcam, Cat #ab 16719) and anti-Rab4 antibody (Cell Signaling Technology, Cat #2167) using biotin-labelled surface protein and whole cell lysate from Kv4.2/KChIP4 transfected HEK293 cell line expressing RyR2 WT or RyR2 E4872Q mutation. Comparison of whole cell I_{A} (N), inactivation time constant Tau (O), V_{A} (P) and V_{H} (Q) recorded in Kv4.2/KCHIP4 transfected HEK293 cell line expressing RyR2 WT (n=16) at baseline, 2 min after application of caffeine (2.5 mM) and 5 min after washout. Comparison of whole cell I_{A} (R), inactivation time constant Tau (S), V_{A} (T) and V_{H} (U) recorded in Kv4.2/KCHIP4 transfected HEK293 cells (n=17) at baseline and 2 min after application of caffeine (2.5 mM). The data shown are the median and range (Kruskal-Wallis test with Dunn-Bonferroni post hoc test; Mann-Whitney U test, paired t test, Wilcoxon matched-pairs signed rank test, repeated measure ANOVA test with Bonferroni post hoc test and Friedman test with Dunn-Bonferroni post hoc test; *P<0.05, **P<0.01 vs WT or baseline, NS, not significant).

FIG. 3 shows that RyR2-E4872Q^{+/-} prevents neuronal hyperactivity of 5×FAD^{+/-} hippocampal CA1 neurons in vivo. Two-photon Ca²⁺ images of hippocampal CA1 region of 5-6 months old WT (A), 5×FAD^{+/-} (B), 5×FAD^{+/-}/EQ^{+/-} (C) and EQ^{+/-} (D) mice in vivo. Colored dots indicate the number of Ca²⁺ transients per minute. (E-H) Ca²⁺ traces of the five neurons circled in A-D, respectively. Histograms showing the frequency distribution of Ca²⁺ transients in (I) WT (8 mice, 2375 cells), (J) 5×FAD^{+/-} (8 mice, 1708 cells), (K) 5×FAD^{+/-}/EQ^{+/-} (8 mice, 2283 cells) and (L) EQ^{+/-} (8 mice, 2392 cells). Pie charts show the relative proportions of silent, normal, and hyperactive neurons as defined previously (Busche, 2018; Busche et al., 2012; Busche et al., 2008). (M) Cumulative probability functions showing frequency distributions of spontaneous Ca²⁺ transients in CA1 region of WT (black), 5×FAD^{+/-} (red), 5×FAD^{+/-}/EQ^{+/-} (green) and EQ^{+/-} (blue) mice (Kruskal-Wallis test with Dunn's multiple comparisons test). (N) Mean Ca²⁺ transient frequency in WT, 5×FAD^{+/-}, 5×FAD^{+/-}/EQ^{+/-} and EQ^{+/-} CA1 region. Percentage of silent (O), normal (P), and hyperactive (Q) cells in WT, 5×FAD^{-/+}, 5×FAD^{+/-}/EQ^{+/-} and EQ^{+/-} CA1 region. Scale bar: 10 μm. Data shown are the median and range (Kruskal-Wallis test with Dunn-Bonferroni post hoc test; *P<0.5, **P<0.01 vs WT, ^{##}P<0.05, ^{##}P<0.01, 5×FAD^{+/-}/EQ^{+/-} vs 5×FAD^{+/-}, NS, not significant).

FIG. 4 shows the effects of RyR2-E4872Q^{+/-} on presynaptic activity and spontaneous AP firing and effects of RyR2-E4872Q^{+/-} and R-carvedilol on RyR2 function. (A) Representative traces of whole-cell voltage-damp recording of spontaneous excitatory post-synaptic currents (sEPSC) in 3-4 months old WT, 5×FAD^{+/-}, 5×FAD^{+/-}/EQ^{+/-} and EQ^{+/-} mouse CA1 neurons. Comparison of the amplitude (B) and inter-event interval (C) of sEPSCs recorded from 3-4 months old WT (6 mice, 11 neurons), 5×FAD^{+/-} (9 mice, 23 neurons), 5×FAD^{+/-}/EQ^{+/-} (6 mice, 15 neurons) and EQ^{+/-} (7 mice, 18 neurons) CA1 neurons. (D) Representative traces of membrane potential recordings showing spontaneous action potential (sAP) firing in 3-4 months old WT, 5×FAD^{+/-}, 5×FAD^{+/-}/EQ^{+/-} and EQ^{+/-} mouse CA1 pyramidal neurons, (E) Fraction of CA1 pyramidal neurons showing sAP firing. (F) sAP firing frequency recorded from WT (6 mice, 21 neurons), 5×FAD^{+/-} (9 mice, 21 neurons), 5×FAD^{+/-}/EQ^{+/-} (6 mice, 18 neurons), and EQ^{+/-} (7 mice, 16 neurons) CA1 neurons. (G) Amplitude of spontaneous Ca²⁺ transients recorded using in vivo Ca²⁺ imaging of CA1 neurons from 5-6 months old WT (8 mice, 2375 cells), 5×FAD^{+/-} (8 mice, 1708 cells), 5×FAD^{+/-}/EQ^{+/-} (8 mice, 2283 cells), and EQ^{+/-} (8 mice, 2392 cells), (H) Caffeine-induced Ca²⁺ release recorded from 3-4 months old GCAMP6f-expressing WT (5 mice, 58 neurons), 5×FAD^{+/-} (5 mice, 67 neurons), 5×FAD^{+/-}/EQ^{+/-} (5 mice, 68 neurons), and EQ^{+/-} (5 mice, 90 neurons) CA1 neurons, showing GCaMP6f images of CA1 pyramidal neurons of different genotypes before (baseline) and after application of caffeine (40 mM) (top left), fluorescence traces (bottom left), and average data (right). (I) Caffeine-induced Ca²⁺ release recorded from 4-5 months old GCAMP6f-expressing 5×FAD^{+/-} mice treated with DMSO (5 mice, 112 neurons) and R-CV (5 mice, 113 neurons) for 1 month, showing GCaMP6f images of CA1 pyramidal neurons treated with DMSO or R-CV before (baseline) and after application of caffeine (40 mM) (top left), fluorescence traces (bottom left), and average data (right). Fraction of cells displaying spontaneous Ca²⁺ oscillation at different external Ca²⁺ concentrations in HEK293 cells expressing RyR2 WI (J) or RyR2-E48720 mutant (K) transfected with control plasmid (pcDNA3), presenilin 1 (PS1)-WT, PS1-M146L or PS1-L286V. Scale bar: 5 μm. The data shown are the median and range (Kruskal-Wallis test with Dunn-Bonferroni post hoc test and Mann-Whitney U test; *P<0.05, **P<0.01 vs WT or control, ^{##}P<0.01, 5×FAD^{+/-}/EQ^{+/-} vs 5×FAD⁺⁻, NS, not significant).

FIG. 5 shows that the RyR2 E4872Q^{+/-} mutation prevents neuronal hyperactivity of 5×FAD^{+/-} hippocampal CA1 neurons in vivo Representative in vivo two-photon Ca²⁺ images of the hippocampal CA1 region of 3-4 months old WT (A), 5× FAD^{+/-} (B), 5×FAD^{+/-}/EQ^{+/-} (C) and EQ^{+/-} (D) mice. The colored dots on the neurons indicate the number of Ca²⁺ transients per minute. (E-H) Ca²⁺ traces of the five neurons circled in A-D, respectively. Histograms showing the frequency distribution of Ca²⁺ transients in WT (I, 7 mice, 1756 cells), 5×FAD^{+/-} (J, 7 mice, 1466 cells), 5×FAD^{+/-}/EQ^{+/-} (K, 6 mice, 1089 cells) and EQ^{+/-} (L, 5 mice, 715 cells). Pie charts show the relative proportions of silent, normal, and hyperactive neurons. (M) Cumulative probability functions showing frequency distributions of spontaneous Ca²⁺ transients in CA1 region of WT (black), 5×FAD^{+/-} (red), 5×FAD^{+/-}/EQ^{+/-} (green) and EQ^{+/-} (blue) mice (Kruskal-Wallis test with Dunn's multiple comparisons test; **P<0.0001 vs WT. ^{##P<}0.0001 5×FAD^{+/31} /EQ^{+/-} 1 vs 5×FAD^{+/-}, NS, not significant). (N) Mean Ca²⁺ transient frequency in WT, 5×FAD^{+/-}, 5×FAD^{+/-}EQ^{+/31} and EQ^{+/-} mice CA1 region as defined previously (Busche, 2018; Busche et al., 2012; Busche et al., 2008). Percentage of silent (O), normal (P) and hyperactive (Q) cells in WT, 5×FAD^{+/-}, 5×FAD^{+/-}/EQ^{+/-} and EQ^{+/-} mice CA1 region. Note that analyses of frequency distributions (I to M) were performed using cells pooled from all animals, while analyses of mean frequency and fraction of silent, normal, and hyperactive cells (N to Q) were based on data from individual animals. Scale bar: 10 μm. The data shown are the median and range (Kruskal-Wallis test with Dunn-Bonferroni post hoc test; *P<0.05, **P<0.01 vs WT, ^{#}P<0.05 5×FAD^{+/-}/EQ^{+/-} vs 5×FAD^{+/-}, NS, not significant).

FIG. 6 shows that the E4872Q^{+/-} mutation prevents memory loss and LTP impairment of 5×FAD^{+/-} mice. (A) The latency to reach the target platform of 5-6 months old WT (n=10), 5×FAD^{+/-} (n=7), 5×FAD^{+/-}/EQ^{+/-} (n=11) and EQ^{+/-} (n=15) mice in the Morris Water Maze (MWM) test. (B) The time spent in the target quadrant. (C) The percentage of time spent on the novel object in the Novel Object Recognition (NOR) test in 5-6 months old WT (n=10), 5×FAD^{+/-} (n=7), 5×FAD^{+/-}/EQ^{+/-} (n=11) and EQ^{+/-} (n=15) mice. (D) The latency to reach the target hole of 10-15 months old WT (n=12), 5×FAD^{+/-} (n=14), 5×FAD^{+/-}/EQ^{+/-} (n=8) and EQ^{+/-} (n=8) mice in the Barnes Maze (BM) test. (E) The number of nose-pokes to each hole on the BM test platform (**5×FAD vs WT). (F) Effect of 100 Hz high frequency stimulation (HFS) on mean Schaffer collateral-evoked fEPSP slope in hippocampal slices from 5-6 months old WT (10 mice, 20 slices), 5×FAD^{+/-} (10 mice, 20 slices), 5×FAD^{+/-}/EQ^{+/-} (10 mice, slices) and EQ^{+/-} (10 mice, 20 slices). (G) The averaged normalized fEPSP slope. (H) Effect of HFS on the fEPSP slope of 10-15 months old WT (10 mice, 20 slices), 5×FAD^{+/-} (10 mice, 20 slices), 5×FAD^{+/-}/EQ^{+/-} (10 mice, 20 slices) and EQ^{+/-} (10 mice, 20 slices). (I) The averaged normalized fEPSP slope. Data shown are the median and range (Kruskal-Wallis test with Dunn-Bonferroni post hoc test; *P<0.5, **P<0.01 vs WT, ^{#}P<0.05, ^{##}P<0.01, 5×FAD^{+/-}/EQ^{+/-} vs 5×FAD^{+/-}, NS, not significant).

FIG. 7 shows that the E4872Q^{+/-} mutation prevents memory loss and LTP impairment in both young and aged 5×FAD^{+/-} mice. (A) The latency to reach the target platform during the training period (days 1-4) of 3-4 months old WT (n=7), 5×FAD^{+/-} (n=7), 5×FAD^{+/-}/EQ^{+/-} (n=7) and EQ^{+/-} (n=7) mice in the Morris Water Maze (MWM) test. (B) The time spent in the target quadrant after removing the platform in the probe test 24 h after the last training session. (C) The percentage of time spent on the novel object during the Novel Object Recognition (NOR) test in 3-4 months old WT (n=7), 5×FAD^{+/-} (n=7), 5×FAD^{+/-}EQ^{+/-} (n=7) and EQ^{+/-} (n=7) mice. (D) The latency to reach the target platform during the training period (days 1-4) of 10-11 months old WT (n=17), 5×FAD^{+/-} (n=15), 5×FAD^{+/31} /EQ^{+/-} (n=14) and EQ^{+/-} (n=15) mice in the Morris Water Maze (MWM) test. (E) The time spent in the target quadrant after removing the platform in the probe test 24 h after the last training session. (F) The percentage of time spent on the novel object during the Novel Object Recognition (NOR) test in 10-11 months old WT (n=17); 5×FAD^{+/-} (n=15), 5×FAD^{+/-}/EQ^{+/-} (n=14) and EQ^{+/-} (n=15) mice. (G) Effect of 100 Hz high frequency stimulation (HFS) on mean CA3-CA1 fEPSP slope in hippocampal slices from 3-4 months old WT (10 mice, 20 slices), 5×FAD^{+/-} (10 mice, 20 slices), 5×FAD^{+/-}/EQ^{+/-} (10 mice, 20 slices) and EQ^{+/-} (10 mice, 20 slices). Data were normalized to the mean fEPSP slope in slices from 20 min baseline recording. (H) The averaged normalized fEPSP slope recorded between 50-60 min after HFS. The data shown are the median and range (Kruskal-Wallis test with Dunn-Bonferroni post hoc test; **P<0.01 vs WT, ^{#}P<0.05, ^{##}P<0.01 5×FAD^{+/-}/EQ^{+/-} vs 5×FAD^{+/-}, NS, not significant).

FIG. 8 shows the effect of the RyR2 E4872Q^{+/-} mutation, carvedilol, and R-carvedilol on the input-output relationship in the mouse hippocampal CA3-CA1 pathway. (A) Representative fEPSP traces recorded from 3-4 months old WT; 5×FAD^{+/-}, 5×FAD^{+/-}/EQ^{+/-} and EQ^{+/-} mouse hippocampal slices before (lighter color) and after (darker color) LTP induction. (B) Input-output relationships between fEPSP slope and stimulus intensity measured from CA3-CA1 pathway in 3-4 months old WT (10 mice, 20 slices), 5×FAD^{+/-} (10 mice, 20 slices), 5×FAD^{+/-}/EQ^{+/-} (10 mice, 20 slices), and EQ^{+/-} (10 mice, 20 slices) mouse hippocampal slices. (C) Representative fEPSP traces recorded from 5-6 months old WT, 5×FAD^{+/-}, 5×FAD^{+/-}/EQ^{+/-} and EQ^{+/-} mouse hippocampal slices before (lighter color) and after (darker color) LTP induction, (D) Input-output relationships between fEPSP slope and stimulus intensity measured from CA3-CA1 pathway in 5-6 months old WT (10 mice, 20 slices), 5×FAD^{+/-} (10 mice, 20 slices), 5×FAD^{+/-}/EQ^{+/-} (10 mice, 20 slices), and EQ^{+/-} (10 mice, 20 slices) mouse hippocampal slices. (E) Representative fEPSP traces recorded from 10-15 months old WT, 5×FAD^{+/-}, 5×FAD^{+/-}/EQ^{+/-}, and EQ^{+/-} mouse hippocampal slices before (lighter color) and after (darker color) LTP induction. (F) Input-output relationships between fEPSP slope and stimulus intensity measured from CA3-CA1 pathway in 10-15 months old WT (10 mice, 20 slices), 5×FAD^{+/-} (10 mice, 20 slices), 5×FAD^{30 /-}/EQ^{+/-} (10 mice, 20 slices), and EQ^{+/-} (10 mice, 20 slices) mouse hippocampal slices. The data shown are the mean±SD. (Two-way ANOVA with Bonferroni post hoc test; **P<0.01 compared to WT, ^{##}P<0.01 5×FAD^{+/-}/EQ^{+/-} vs 5×FAD^{+/-}). (G) Representative fEPSP traces recorded from hippocampal slices of 3-4 months old 5×FAD^{+/-} mice treated with DMSO or R-carvedilol (R-CV) before (lighter color) and after (darker color) LTP induction. (H) Input-output relationships between fEPSP slope and stimulus intensity measured from hippocampal slices of 3-4 months old 5×FAD^{+/-} mice treated with DMSO (10 mice, 20 slices) or R-CV (10 mice, 20 slices). (I) Representative fEPSP traces recorded from hippocampal slices of 4-5 months old 5×FAD^{+/-} mice treated with DMSO or R-CV before (lighter color) and after (darker color) LTP induction. (J) Input-output relationships between fEPSP slope and stimulus intensity measured from hippocampal slices of 4-5 months old 5×FAD^{+/-} mice treated with DMSO (10 mice, 20 slices) or R-CV (10 mice, 20 slices). (K) Representative fEPSP traces recorded from hippocampal slices of 6-7 months old 5×FAD^{+/-} mice treated with DMSO or R-CV before (lighter color) and after (darker color) LTP induction. (L) Input-output relationships between fEPSP slope and stimulus intensity measured from hippocampal slices of 6-7 months old 5×FAD^{+/-} mice treated with DMSO (10 mice, 20 slices) or R-CV (10 mice, 20 slices). (M) Representative fEPSP traces recorded from hippocampal slices of 3-4 months old 5×FAD^{+/-} mice treated with DMSO or carvedilol before (lighter color) and after (darker color) LTP induction. (N) Input-output relationships between fEPSP slope and stimulus intensity measured from hippocampal slices of 3-4 months old 5×FAD^{+/-} mice treated with DMSO (10 mice, 20 slices) or carvedilol (10 mice, 20 slices). The data shown are the mean±SD (Two-way ANOVA with Bonferroni post hoc test; **P<0.01 compared to the DMSO group).

FIG. 9 shows that R-carvedilol prevents and rescues neuronal hyperactivity of 5×FAD^{+/-} hippocampal CA1 neurons in vivo. Two-photon in vivo Ca²⁺ imaging of hippocampal CA1 region of 3-4 months (A, B) or 4-5 months (G, H) old 5×FAD^{+/-} mice treated with vehicle control (DMSO) (A, G) or R-carvedilol (R-CV) (3.2 mg/kg/day) (B, H). Colored dots indicate the number of Ca²⁺ transients per minute. (C, D, I, J) Ca²⁺ traces of the five neurons circled in A, B, G, and H, respectively. Histograms showing the frequency distribution of Ca²⁺ transients in DMSO treated (E, 5 mice, 1066 cells, and K, 5 mice, 897 cells), and R-CV treated (F, 5 mice, 1181 cells, and L, 5 mice, 757 cells). Pie charts show the relative proportions of silent, normal, and hyperactive neurons. (M) Cumulative probability functions showing frequency distributions of spontaneous Ca²⁺ transients in CA1 region of 3-4 months old 5×FAD^{+/-} mice treated with DMSO (red); R-CV (blue) and 5-6 months old 5×FAD^{+/-} mice treated with DMSO (black), R-CV (green) (Kruskal-Wallis test with Dunn's multiple comparisons test). (N) Mean Ca²⁺ transient frequency in CA1 region of 3-4 months old and 4-5 months old 5×FAD^{+/-} mice treated with DMSO or R-CV. Percentage of silent (O), normal (P) and hyperactive (Q) in CA1 region of 3-4 and 4-5 months old 5×FAD^{+/-} mice treated with DMSO or R-CV. Scale bar: 10 μm. Data shown are the median and range (Kolmagorov-Smirnov test with Dunn-Bonferroni post hoc test and Mann-Whitney U test; **P<0.01 vs Control, NS, not significant).

FIG. 10 shows that R-carvedilol prevents and rescues memory loss and LTP impairment in 5×FAD^{+/-} mice. The latency to reach the target platform of 3-4 (A) and 4-5 (F) months old 5×FAD^{+/-} mice treated with DMSO (3-4 months, n=11, and 4-5 months, n=8) or R-carvedilol (R-CV) (3-4 m, n=13, and 4-5 m, n=10) in the MWM test. (B, G) The time spent in the target quadrant. (C, H) The percentage of time spent on the novel object in 5×FAD^{+/-} mice treated with DMSO (3-4 m, n=11, and 4-5 m, n=8) or R-CV (3-4 m, n=13 and 4-5 m, n=10). (D, I) Effect of HFS on mean CA3-CA1 fEPSP slope in hippocampal slices from 5×FAD^{+/-} mice treated with DMSO (3-4 m, 10 mice, 20 slices, and 4-5 m, 10 mice, 20 slices) or R-CV (3-4m, 10 mice, 20 slices, and 4-5m, 10 mice, 20 slices). (E, J) The averaged normalized fEPSP slope of 3-4 (E) or 4-5 months (J) old 5×FAD^{+/-} mice treated with DMSO or R-CV. Data shown are the median and range (Mann-Whitney U test; *P<0.5, **P<0.01 compared to the DMSO group).

FIG. 11 shows that R-carvedilol but not carvedilol racemic mixture rescues memory loss and LTP deficit in 5×FAD^{+/- mice.} 5×FAD^{+/-} mice were treated with DMSO or R-carvedilol (R-CV, 3.2 mg/kg/day) for 1 month, starting at 5-6 months or 9-11 months of age after the onset of neuronal hyperactivity and memory loss. (A) The latency to reach the target platform during the training period (days 1-4) of 6-7 months old 5×FAD^{+/-} mice after 1-month treatment with DMSO (n=6) or R-CV (n=7) in MWM test. (B) Comparison of the time spent in the target quadrant after removing the platform in the probe test 24 h after the last training session. (C) Comparison of the percentage of time spent on the novel object during the NOR test in 6-7 months old 5×FAD^{+/-} mice treated with DMSO (n=6) or R-CV (n=7). (D) Effect of 100 Hz high frequency stimulation (HFS) on mean CA3-CA1 fEPSP slope in brain slices from 6-7 months old 5×FAD^{+/-} mice treated with DMSO (10 mice, 20 slices) or R-CV (10 mice, 20 slices). (E) Comparison of the averaged normalized fEPSP slop recorded between 50-60 min after HFS from 6-7 months old 5×FAD^{+/-} mice treated with DMSO or R-CV. (F) The latency to reach the target platform during the training period (days 1-4) of 10-12 months old 5×FAD^{+/-} mice after 1-month treatment with DMSO (n=13) or R-CV (n=12) in MWM test. (G) Comparison of the time spent in the target quadrant after removing the platform in the probe test 24 h after the last training session. (H) Comparison of the percentage of time spent on the novel object during the NOR test in 10-12 months old 5×FAD^{+/-} mice treated with DMSO (n=8) or R-CV (n=5). 5×FAD^{+/-} mice were treated with DMSO or carvedilol (3.2 mg/kg/day) for 1 month, starting at 2-3 months of age before the onset of neuronal hyperactivity and memory loss. (I) The latency to reach the target platform during the training period (days 1-4) of 3-4 months old 5×FAD^{+/-} mice after 1-month treatment with DMSO (n=11) or carvedilol (n=7) in MWM test. (J) Comparison of the time spent in the target quadrant after removing the platform in the probe test 24 h after the last training session. (K) Comparison of the percentage of time spent on the novel object during the NOR test in 3-4 months old 5×FAD^{+/-} mice treated with DMSO (n=11) or carvedilol (n=7). (L) Effect of 100 Hz high frequency stimulation (HFS) on mean CA3-CA1 fEPSP slope in brain slices from 3-4 months old 5×FAD^{+/-} mice treated with DMSO (10 mice, 20 slices) or carvedilol (10 mice, 20 slices). (M) Comparison of the averaged normalized fEPSP slop recorded between 50-60 min after HFS from 3-4 months old 5×FAD^{+/-} mice treated with DMSO or carvedilol. The data shown are the median and range (Mann-Whitney U test; *P<0.05, **P<0.01 vs DMSO group, NS, not significant).

FIG. 12 shows that R-carvedilol rescues memory loss in 5×FAD^{+/-} mice in a dose-dependent manner. The latency to reach the target platform of 4-6 months old 5×FAD^{+/-} mice treated for one month with DMSO (n=7), R-carvedilol (3.2 mg/kg/day) (n=7), R-carvedilol (1.6 mg/kg/day) (n=9), and R-carvedilol (0.8 mg/kg/day) (n=9) in the Morris Water Maze (MWM) test (A). The time spent in the target quadrant in the MWM test (B). The percentage of time spent on the novel object of 5×FAD^{+/-} mice treated for one month with DMSO (n=7), R-carvedilol (3.2 mg/kg/day) (n=7), R-carvedilol (1.6 mg/kg/day) (n=9), and R-carvedilol (0.8 mg/kg/day) (n=7) in the Novel Objective Recognition (NOR) test (C). Data shown are mean±SEM (*P<0.05, **P<0.01 compared to the DMSO group, NS, not significant).

FIG. 13 shows that E4872Q^{+/-} has no significant impact on Aβ-accumulation but protects against neuron loss in 5×FAD^{+/-} mice. (A) Aβ deposition in 10-15 months old WT, EQ^{+/-}, 5×FAD^{+/-} and 5×FAD^{+/-}/EQ^{+/-} hippocampal region. (B) Averaged Aβ plaque number in 10-15 months old 5×FAD^{+/-} (12 mice, 36 slices) and 5×FAD^{+/-}/EQ^{+/-} (12 mice, 36 slices) hippocampal region. (C) Percentage of hippocampal area showing positive Aβ staining. (D) Immunoblotting analysis of brain homogenates from 10-15 months old WT, EQ^{+/-}, 5×FAD^{+/-}, and 5×FAD^{+/-}/EQ^{+/-} mice. (E) Normalized total Aβ level and (F) Normalized Aβ (1-42) levels in WT (n=15), EQ^{+/-} (n=11), 5×FAD^{+/-} (n=21), and 5×FAD^{+/-}/EQ^{+/-} (n=20) brains. (G) Images of Nissl staining of 10-15 months old WT, EQ^{+/-}, 5×FAD^{+/-} and 5×FAD^{+/-}/EQ^{+/-} brain sections. (H) Numbers of pyramidal neurons in the subiculum region (red oval) of WT (12 mice, 36 slices), EQ^{+/-} (12 mice, 36 slices), 5×FAD^{+/-} (12 mice, 36 slices) and 5×FAD^{+/-}/EQ^{+/-} (12 mice, 36 slices). (I) Images of Nissl staining of 10-15 months old WT, EQ^{+/-}, 5×FAD^{+/-} and 5×FAD^{+/-}/EQ^{+/-} brain sections. (J) Numbers of pyramidal neurons in the CA1 region of WT (12 mice, 36 slices), EQ^{+/-} (12 mice, 36 slices), 5×FAD^{+/-} (12 mice, 36 slices) and 5×FAD^{+/-}/EQ^{+/-} (12 mice, 36 slices). Data shown are the median and range (Mann-Whitney U test and Kruskal-Wallis test with Dunn-Bonferroni post hoc test; **P<0.01 vs WT, ^{##P<}0.01, 5×FAD^{+/-}/EQ^{+/-} vs 5×FAD^{+/-}, NS, not significant).

FIG. 14 shows the effect of the E4872Q^{+/-} mutation and R-carvedilol on Aβ-accumulation and effect of E4872Q^{+/-} on CA1 pyramidal neuron apical dendritic spine density and morphology. (A) Aβ deposition in the hippocampus in 5-6 months old WT, EQ^{+/-}, 5×FAD^{+/-} and 5×FAD^{+/-}/EQ^{+/-} mouse brains. Sagittal brain sections were stained with anti-Aβ (total) antibody (Cell signaling Technology, Cat #8243) and the bound antibodies were visualized using DAB staining, No plaque was detected in WT or EQ mice. (B) Averaged Aβ plaque number per mm 2 in 5-6 months old 5×FAD^{+/-} (12 mice, 36 slices) and 5×FAD^{+/-}/EQ^{+/-} (12 mice, 36 slices) mouse hippocampal region. (C) Percentage of hippocampal area showing positive Aβ staining. (D) Immunoblotting analysis with anti-Aβ (total) antibody (Cell Signaling Technology, Cat #8243) or anti-Aβ (1-42) antibody (Biolegend, Cat #803001) using brain homogenates from 5-6 months old WT, EQ^{+/-}, 5×FAD^{+/-}, and 5×FAD^{+/-}/EQ^{+/-} mice. (E) Normalized total Aβ levels and (F) Normalized Aβ (1-42) levels in WT (n=9), EQ^{+/-} (n=12), 5×FAD^{+/-} (n=11), and 5×FAD^{+/-}/EQ^{+/-} (n=14) brains. Note that there are no Aβ-specific signals detected in the WT or EQ^{+/-} brain tissue homogenates, as expected. (G-L) 5×FAD^{+/-} mice were treated with DMSO or R-carvedilol (R-CV) (3.2 mg/kg/day) for 1 month, starting at 2-3 months or 3-4 months of age before or after the onset of neuronal hyperactivity and memory loss. (G) Immunoblotting analysis with anti-Aβ (total) antibody (Cell Signaling Technology, Cat #8243) or anti-Aβ (1-42) antibody (Biolegend, Cat #803001) using brain tissue homogenates from 3-4 months old 5×FAD^{+/-} mice after 1-month treatment with DMSO or R-CV. (H) Normalized total Aβ levels and (I) Normalized Aβ (1-42) levels in 5×FAD^{+/-} mice treated with DMSO (n=13) or R-CV(n=13) at the age of 3-4 months old. (J) Immunoblotting using brain tissue homogenates from 4-5 months old 5×FAD^{+/-} mice after 1-month treatment with DMSO or R-CV. (K) Normalized total Aβ levels and (L) Normalized Aβ (1-42) levels in 5×FAD^{+/-} mice treated with DMSO (n=11) or R-CV (n=11) at the age of 4-5 months old. The data shown are the median and range (Mann-Whitney U test; NS, not significant). (M) Apical dendritic spine density analyzed from 9-12 months old WT (3 mice, 27 dendrites), 5×FAD^{+/-} (3 mice, 27 dendrites), 5×FAD^{+/-}/EQ^{+/-} (3 mice, 27 dendrites), and EQ^{+/-} (3 mice, 27 dendrites) CA1 neurons. Upper panel: representative Golgi staining images, scale bar: 10 μm. Lower panel: densities of overall protrusions and different types of dendritic spines. The data shown are the median and range (Kruskal-Wallis test with Dunn-Bonferroni post hoc test; *P<0.05, **P<0.01 vs WT, ^{#}P<0.05, ^{##}P<0.01 5×FAD^{+/-}/EQ^{+/-} vs 5×FAD^{+/-}, NS, not significant).

FIG. 15 shows that R-carvedilol treatment has no significant impact on Aβ-accumulation but protects against neuron loss in 6-7 months old 5×FAD^{+/-} mice. (A) Aβ deposition in 6-7 months old 5×FAD^{+/-} mice treated with DMSO or R-CV. (B) Averaged Aβ plaque number in 6-7 months old 5×FAD^{+/-} mouse hippocampal region treated with DMSO (12 mice, 36 slices) or R-CV (12 mice, 36 slices). (C) Percentage of hippocampal area showing positive Aβ staining. (D) Immunoblotting analysis of brain tissue homogenates from 6-7 months old 5×FAD^{+/-} mice treated with DMSO or R-CV, (E) Normalized total Aβ levels and (F) Normalized Aβ (1-42) levels in 5×FAD^{+/-} mice treated with DMSO (n=10) or R-CV (n=11). (G) Images of Nissl staining of 6-7 months old 5×FAD^{+/-} mice treated with DMSO or R-CV. (H) Numbers of pyramidal neurons in the subiculum region (red oval) of 6-7 months old 5×FAD^{+/-} mice treated with DMSO (12 mice, 36 slices) or R-CV (12 mice, 36 slices). (I) Images of Nissl staining of 6-7 months old 5×FAD^{+/-} mice treated with DMSO or R-CV. (J) Numbers of pyramidal neurons in the CA1 region of 6-7 months old 5×FAD^{+/-} mice treated with DMSO (12 mice, 36 slices) or R-CV (12 mice, 36 slices). Data shown are the median and range (Mann-Whitney U test; **P<0.01 compared to the DMSO group, NS, not significant).

FIG. 16 shows that the RyR2 E487 2Q^{+/-} mutation prevents memory loss of 3×TG^{+/-} mice. (A) The latency to reach the target platform of 12-15 months old WT (n=12), 3×TG^{+/-} (n=10), 3×TG^{+/31} /EQ^{+/-} (n=10) and EQ^{+/- (n=}11) mice in the Morris Water Maze (MWM) test. Kruskal-Wallis test with Dunn-Bonferroni post hoc test was used for data points from the same training day. (B) The time spent in the target quadrant. (C) Average swimming speed in water maze test. (D) The percentage of time spent on the novel object in the Novel Object Recognition (NOR) test in 12-15 months old WT (n=11), 3×TG^{+/-} (n=10), 3×TG^{+/-}/EQ^{+/-} (n=10) and EQ^{+/-} (n=8) mice. (E) Average walking velocity during the habituation phase in the NOR test. (F) The latency to reach the target hole of 12-15 months old WT (n=9), 3×TG^{+/-} (n=12)), 3×TG^{+/-}EQ^{+/-} (n=10) and EQ^{+/-} ((n=7) mice in the Barnes Maze (BM) test. Kruskal-Wallis test with Dunn-Bonferroni post hoc test was used for data points from the same training day. (G) The number of nose-pokes to each hole on the BM test platform (**3×TG^{+/-} vs WT). Data shown are the median and range (Kruskal-Wallis test with Dunn-Bonferroni post hoc test; *P<0.05, **P<0.01 vs WT, ^{##}P<0.01, 3×TG^{+/-}EQ^{+/-} vs 3×TG^{+/-}, NS, not significant).

FIG. 17 shows that the E4872Q^{+/-} mutation prevents LTP deficit of 3×TG^{+/-} mice. (A) Effect of 100 Hz high frequency stimulation (HFS) on mean Schaffer collateral-evoked fEPSP slope in hippocampal slices from 12-15 months old WT (6 mice, 12 slices), 3×TG+/− (5 mice, 13 slices), 3×TG^{+/-}/EQ^{+/-} (5 mice, 13 slices) and EQ^{30 /-} (6 mice, 12 slices). (B) The average normalized fEPSP slope. Data shown are the median and range (Kruskal-Wallis test with Dunn-Bonferroni post hoc test; **P<0.01 vs WT, ^{##}P<0.01, 3×TG^{+/-}/EQ^{+/-} vs 3×TG^{+/-}, NS, not significant).

FIG. 18 shows the effect of the E4872Q^{+/-} mutation on CA1 pyramidal neuron apical dendritic spine density and morphology. (A) Representative Golgi staining images of dendritic segments from WT, 3×TG^{+/-}, 3×TG^{+/-}/ EQ^{+/-}, and EQ^{+/-} hippocampal CA1 neurons. Note that the images shown were taken at a single Z-(focal) plane, in which some of the spines were out of focus. Scale bar: 5 μm for all 4 images. (B) Densities of overall protrusions and different types of dendritic spines in 12-15 months old WT (4 mice, 15 dendrites), 3×TG^{+/-} (4 mice, 15 dendrites), 3×TG^{+/-}/EQ^{+/-} (4 mice, 15 dendrites), and EQ^{+/-} (4 mice, 15 dendrites) CA1 neurons. A series of Z-stack images of the Golgi-stained apical dendrites of CA1 neurons were used to reconstruct the three-dimensional (3D) dendritic segments using ImageJ and the RECONSTRUCT program (Risher et al., 2014). Different types of spine (filopodia, long thin, thin, stubby, mushroom and branched spines) as defined previously by Risher et al (Risher et al., 2014) can be clearly identified from the reconstructed 3D dendritic segments. Data shown are the median and range (Kruskal-Wallis test with Dunn-Bonferroni post hoc test; *P<0.05, **P<0.01 vs WT, ^{##}P<0.01 3×TG^{+/-}/EQ^{+/-} vs 3×TG^{+/-}, NS, not significant).

FIG. 19 shows that E4872Q^{+/-} protects against neuron loss in the subiculum region in 3×TG^{+/-} mice. (A) Images of Nissl staining of 12-15 months old WT, EQ^{+/-}, 3×TG^{+/-} and 3×TG^{+/-}/EQ^{+/-} brain sections. (B) Numbers of pyramidal neurons in the CA1 region of WT (4 mice, 12 slices), EQ^{+/-} (4 mice, 12 slices), 3×TG^{+/-} (4 mice, 12 slices) and 3×TG^{+/-}/EQ^{+/-} (4 mice, 12 slices). (C) Images of Nissl staining of 12-15 months old WT, EQ^{+/-}, 3×TG^{+/-} and 3×TG^{+/-}/EQ^{+/-} brain sections. (D) Numbers of pyramidal neurons in the subiculum region (red oval) of WT (4 mice, 12 slices), EQ^{+/-} (4 mice, 12 slices), 3×TG^{+/-} (4 mice, 12 slices) and 3×TG^{+/-}/EQ^{+/-} (4 mice, 12 slices). Scale bar: 200 μm in (A) and (C). (E) Aβ deposition in the hippocampus in 12-15 months old WT, EQ^{+/-}, 3×TG^{+/-} and 3×TG^{+/-}/EQ^{+/-} mouse brains. Sagittal brain sections were stained with anti-Aβ (total) antibody (Cell signaling Technology, Cat #8243) and the bound antibodies were visualized using DAB staining. No plaque was detected in WT or EQ^{+/-} mouse brains. (F) Average Aβ plaque number per mm² in 12-15 months old 3×TG^{+/-} (7 mice, 21 slices) and 3×TG^{+/-}EQ^{+/-} (7 mice, 21 slices) mouse hippocampal region. (G) Percentage of hippocampal area showing positive Aβ staining. (H) Immunoblotting analysis with anti-RyR2 antibody (Alomone Labs, Cat #ARR-002) using brain homogenates from 12-15 months old WT, EQ^{+/-}, 3×TG^{+/-}, and 3×TG^{+/-}/EQ^{+/-} mice. (I) Normalized RyR2 levels in WT (n=6), EQ^{+/-} (n=6), 3×TG^{+/-} (n=6), and 3×TG^{+/-}/EQ^{+/-} (n=6) brains. Data shown are the median and range (Mann-Whitney U test and Kruskal-Wallis test with Dunn-Bonferroni post hoc test; **P<0.01 vs WT, #P<0.05, 3×TG^{+/-}EQ^{+/-} vs 3×TG^{+/-}, NS, not significant).

FIG. 20 shows that R (+)-carvedilol rescues memory impairment and LTP deficit in 3×TG^{+/-} mice. (A) The latency to reach the target platform of 12-15 months old 3×TG^{+/-} mice treated with DMSO (n=12) or R-carvedilol (R-CV) (n=11) in the MWM test, Mann-Whitney U test was used for data points from the same training day. (B) The time spent in the target quadrant. (C) Average swimming speed in water maze test. (D) The percentage of time spent on the novel object in 3×TG^{+/-} mice treated with DMSO (n=8) or R-CV (n=7). (E) Average walking velocity during the habituation phase in the NOR test. (F) Mean CA3-CA1 fEPSP slope in hippocampal slices from 12-15 months old 3×TG^{+/-} mice treated with DMSO (5 mice, 9 slices) or R-CV (6 mice, 11 slices). (G) Average normalized fEPSP slopes of 12-15 months old 3×TG^{+/-} mice treated with DMSO or R-CV. Data shown are the median and range (Mann-Whitney U test; *P<0.05, **P<0.01 compared to the DMSO group).

FIG. 21 shows that R (+)-carvedilol rescues neuron loss but not Aβ accumulation in 3×TG^{+/-} mice. (A) Images of Nissl staining of 12-15 months old 3×TG^{+/-} mice treated with DMSO or R-CV. (B) Numbers of pyramidal neurons in the CA1 region of 12-15 months old 3×TG^{+/-} mice treated with DMSO (4 mice, 12 slices) or R-CV (4 mice, 12 slices). (C) Images of Nissl staining of 12-15 months old 3×TG^{+/-} mice treated with DMSO or R-CV. (D) Numbers of pyramidal neurons in the subiculum region (red oval) of 12-15 months old 3×TG^{+/-} mice treated with DMSO (4 mice, 12 slices) or R-CV (4 mice, 12 slices). Scale bar: 100 μm in (A) and (C). (E) Aβ deposition in the hippocampus in 12-15 months old 3×TG^{+/-} mice after 1-month treatment with DMSO or R-CV. Sagittal brain sections were stained with anti-Aβ (total) antibody (Cell signaling Technology, Cat #8243) and the bound antibodies were visualized using DAB staining. (F) Average Aβ plaque number per mm² in 12-15 months old 3×TG^{+/-} mouse hippocampal region after 1-month treatment with DMSO (7 mice, 21 slices) or R-CV (7 mice, 21 slices). (G) Percentage of hippocampal area showing positive Aβ staining. Data shown are the median and range (Mann-Whitney U test; *P<0.05, **P<0.01 compared to the DMSA group).

### DETAILED DESCRIPTION OF THE INVENTION

### Definitions

Unless defined otherwise, all technical and scientific terms used herein have the same meaning as commonly understood by one of ordinary skill in the art to which this invention pertains. A singular expression may include a plural expression unless they are definitely different in a context.

The term “Alzheimer's Disease”, also referred to as AD, as used herein encompasses both familial and sporadic Alzheimer's Disease, early onset Alzheimer's Disease and late onset disease and includes mixed dementia having an Alzheimer's Disease component. Methods of identifying subjects with Alzheimer's Disease or suspected of having Alzheimer's Disease are known in the art. The term also includes early stages including those stages with no significant or minimal cognitive decline and memory loss and late and end stage Alzheimer's Disease. Alzheimer's Disease also includes asymptotic subjects identified using methods known in the art including MRI, optical coherence tomography angiography as having hallmarks of Alzheimer's Disease, Alzheimer's Disease also includes diseases having the hallmarks of human Alzheimer's Disease in non-human animals.

The term “Alzheimer's Dementia” refers to a patient diagnosed with “Alzheimer's Disease” having at least one symptom of dementia including memory loss, both short-term and long-term, and cognitive difficulties. Alzheimer's Dementia also includes diseases having the hallmarks of human Alzheimer's Dementia in non-human animals.

The term “R-carvedilol” as used herein refers to R-(+)-carvedilol substantially free of the S-(−)-carvedilol.

The expression “substantially free of the S-(−)-carvedilol” as used herein means less than 5%, 3%, 1%, 0.5%, 0.1% and 0.01% of the S-(−)-carvedilol by weight.

The term “subject” or “patient” as used herein refers to an animal in need of treatment.

The term “animal,” as used herein, refers to both human and non-human animals, including, but not limited to, mammals.

The term “therapeutically effective amount” as used herein refers to the amount of an active agent that is nontoxic but sufficient to provide the desired therapeutic effect. The amount that is “effective” will vary from subject to subject, depending on the age and general condition of the individual, the particular active agent or agents, and the like as known to those skilled in the art.

The terms “treat,” “treating”, “treatment” or the like refer to an intervention performed with the intention of improving a subject's status. Subjects in need of treatment are those already diagnosed as having Alzheimer's Disease as well as those suspected of having same though no formal diagnosis has been made. The improvement can be subjective or objective and is related to the amelioration of the symptoms associated with, preventing the further development or progression of, or altering the pathology of Alzheimer's Disease. These terms are intended to encompass “improving outcomes” such as “improving quality of life” “extending the life”, and “improving clinical outcomes.” The terms also encompass moderation, reduction, and curing of Alzheimer's Disease at various stages. Examples include prevention of deterioration of a subject's status; and arresting or delaying progression through clinically recognized stages of a disease, such as progression from pre-clinical disease to clinical disease.

As used herein, the expressions “prevent,” “preventing,” “prevention”, or the like means a preventive or prophylactic treatment performed with the intention of preventing or delaying the onset of Alzheimer's Disease.

The term “ameliorate” or “amelioration” includes the arrest, prevention, decrease, or improvement in one or more symptoms, signs, and features of Alzheimer's Disease, either temporary or long-term,

As used herein, the term “comprising” which is synonymous with “including,” “containing”, “having” or “characterized by,” is inclusive or open-ended and does not exclude additional, unrecited elements, ingredients, or method steps.

As used herein, the term “consisting of” excludes any element, step, or ingredient not specified.

As used herein, the term “consisting essentially of” excludes any element, step, or ingredient not specified except for those that do not materially affect the basic and novel characteristic(s) of the invention relating to treatment or prevention of Alzheimer's Disease.

As used herein, the terms “about” or “approximately” when applied to a particular value (e.g. “about 200° C.” or “approximately 200° C.”) or to a range (e.g. “about x to approximately y”) means the value or range includes variation caused by a variety of factors, e.g. the method used to measure an amount, and is no more than ±5% of the value or range. It is to be understood that such a variation is always included in any given value provided herein, whether or not it is specifically referred to.

The ranges of values recited herein are intended to include all values within the ranges. Thus, for example, a range of about 1.6 mg to about 50 mg daily is intended to cover from about 1.7, 1.8, 1.9, 2, 3, or 4, etc., and up to about 49.9, 49.8, 49, 48, 47, 30, or 20, etc. mg daily.

Surprisingly, the inventors have found that the administration of R-carvedilol in two mouse models of Alzheimer's Disease was effective to ameliorate Alzheimer's Disease associated neuronal hyperactivity, impairment of long-term potentiation, and neuronal cell death and was effective at preventing memory loss and learning deficits in these mice. Also surprisingly, these effects were achieved without preventing accumulation of beta-amyloid plaques in these mouse models.

The present invention provides methods of treating or delaying the progression of a Alzheimer's Disease, using a therapeutically effective amount of R-carvedilol. The treatment of Alzheimer's Disease includes alleviating or reducing at least one adverse or negative effect or symptom, including memory loss, cognitive difficulties, confusion, loss of bladder and bowel control, etc.

As some studies suggest, neuronal hyperactivity may be an early primary dysfunction in AD in humans and animal models. The invention provides neuronal hyperactivity-directed therapeutics and therapeutic methods based on a previously unknown mode of ryanodine receptor 2 (RyR2) control of neuronal hyperactivity.

In some embodiments, the methods of the invention are based on the finding that a single RyR2 point mutation E4872Q, which reduces RyR2 open time, prevents neuronal hyperactivity, impairment of long-term potentiation, memory impairment, neuronal cell death, and dendritic spine loss in a severe, early-onset AD mouse model (5×FAD). Accordingly, the invention provides methods of treating or preventing Alzheimer's Disease by limiting ryanodine receptor type 2 open time. In particular embodiments, the invention provides methods of pharmacologically limiting RyR2 open time with the R-carvedilol enantiomer to prevent and/or rescue neuronal hyperactivity, impairment of long-term potentiation, memory impairment, and/or neuron loss in mouse models of AD.

In some embodiments relating to AD, the neuronal hyperactivity-directed therapeutics and therapeutic methods prevent or delay AD progression including progression from pre-dementia to early AD, early to moderate and moderate to advanced.

### R-Carvedilol and Compositions

R-carvedilol (whose chemical structure is shown below) was shown in a study (Zhang et al., 2015) to limit the open time of cardiac RyR2 channels.

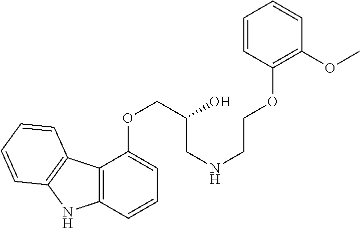 [SMILES: COc1ccccc1OCCNC[C@@H](O)COc1cccc2[nH]c3ccccc3c12]

Racemic carvedilol is a non-selective beta- and alpha-adrenergic receptor blocker that has also previously been identified as a small molecule inhibitor of RyR2 present in cardiac tissue. R and S enantiomers of carvedilol have different activities. Unlike the S enantiomer, the R enantiomer is non-beta-blocking.

The R enantiomer is an alpha-adrenergic receptor blocker and acts directly on RyR2 to reduce the open duration of the cardiac ryanodine receptor (RyR2) Ca²⁺ release channel, and suppresses the store-overload-induced Ca²⁺ release. R-carvedilol as used herein refers to the R enantiomer of carvedilol substantially free of the S enantiomer of carvedilol and compositions comprising the same that are substantially free of the S carvedilol enantiomer associated beta blocking activity.

In some embodiments, R-carvedilol has an optical purity by weight of at least 95%, at least 97%, at least 99%, at least 99.5%, at least 99.9% or at least 99.99%. In other embodiments, R-carvedilol is optically pure.

R-carvedilol, and metabolites of or pharmaceutically acceptable salts or solvates thereof, can be prepared by methods known to those of ordinary skill in the art. For example, in some embodiments, R-carvedilol is prepared from S-(−)-glycidol as described below or as described in U.S. Pat. No. 8,101,781.

R-carvedilol may be provided in a pharmaceutical composition. The pharmaceutical composition may contain additives such as binders, plasticizers, diluents, carriers, glidants, excipients, antistatics, adsorbing agents, separating agents, dispersants, drageeing lacquers, de-foamers, film formers, emulsifiers, disintegrants and fillers in the tablets and/or the coating, Tablets or granulates, for example, can contain flavor-improving additives as well as substances usually used as preservatives, stabilizers, moisture-retainers and emulsifiers, salts for varying the osmotic pressure, buffers and other additives.

Pharmaceutically acceptable carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions as described herein may be sterilized by conventional methods and/or lyophilized.

Methods of improving R-carvedilol solubility are known in the art and include the use of solubility enhancement agents including cyclodextrin, solid dispersion methods, use of a polyoxyethylene-polyoxypropylene copolymer as a surfactant, microwave methods amongst other well-known methods.

R-carvedilol may be by any suitable means or by any suitable route.

In some embodiments, the R-carvedilol is formulated for oral administration or for parenteral administration including for subcutaneous, intramuscular, or intravenous administration.

In some embodiments of the invention, the pharmaceutical compositions are formulated as a nasal spray, aerosol or nasal drop.

In some embodiments, the R-carvedilol may be provided as part of a controlled release formulation.

Optionally, R-carvedilol is the only pharmaceutically active ingredient in the composition. Alternatively, additional pharmaceutically active ingredients may be included.

Metabolites include those that can suppress store overload-induced calcium release such as those described in Malig et al, (2016) and U.S. Pat. No. 6,358,990B1.

In some embodiments, the metabolite is 3-hydroxycarvedilol and/or 4′-hydroxycarvedilol and/or 5′-hydroxycarvedilol.

### Methods and Uses of the Invention

R-carvedilol can be used in methods of treating or preventing Alzheimer's Disease in a subject in need thereof. The methods include administering a therapeutically effective amount of R-carvedilol, a metabolite of R-carvedilol, or a salt thereof, optionally provided as a pharmaceutical composition, to a subject having or a subject at risk of developing Alzheimer's Disease.

In some embodiments, the Alzheimer's Disease is preclinical Alzheimer's Disease, Mild cognitive impairment, or Alzheimer's dementia.

The methods of the invention can be used to prevent Alzheimer's Disease or progression of Alzheimer's Disease from pre-clinical or early-stages to middle or late-stage disease. In some embodiments, methods of the invention can be used to stabilize a subject that with Alzheimer's Disease.

In some embodiments, the methods of the invention can be used to ameliorate one or more symptoms of Alzheimer's Disease, Accordingly, in some embodiments, the methods of the invention can rescue and/or at least partially restore memory in a subject with Alzheimer's Disease, prevent and/or mitigate hippocampal long-term potentiation impairment in the subject with Alzheimer's Disease, prevent and/or mitigate neuronal hyperactivity in a subject with Alzheimer's Disease, prevent or mitigate memory loss in a subject with Alzheimer's Disease, and/or prevent or mitigate cognitive decline in a subject with Alzheimer's Disease including cognitive decline relating to one or more of the following: spatial awareness, exploration, associative memory, working memory and reference memory.

In some embodiments, the methods of the invention can be used to prevent or mitigate neuronal cell death in a subject with Alzheimer's Disease,

In some embodiments, the methods of the invention can be used to prevent or mitigate dendritic spine loss in a subject with Alzheimer's Disease.

In some embodiments, the methods of the invention can be used to prevent or mitigate learning deficits in a subject with Alzheimer's Disease.

The methods of the invention can be used to prevent cognitive decline in a subject identified as being at risk of developing Alzheimer's Disease. Individuals can be identified as being at risk of developing Alzheimer's based on family history of Alzheimer's Disease, previous head trauma, presence of brain abnormalities and presence of genetic risk factors amongst other risk factors known in the art.

Optionally, the methods of the invention can be used in combination with other therapies to treat or prevent Alzheimer's Disease or a symptom thereof. In some embodiments, the other therapies include of a therapeutically effect amount of at least one other therapeutic agent,

In some embodiments, the other therapeutic agent is a cholinesterase inhibitor including Donepezil, Galantamine and Rivastigmine, an anti-tau therapy, anti- beta-amyloid therapy including Aducanumab, a N-methyl-D-aspartate receptor antagonist including Memantine or a therapeutic agent for treating a symptom of Alzheimer's Disease.

In some embodiments, the other therapeutic agent is for treating a symptom of Alzheimer's Disease and may include antidepressants and/or antipsychotics and/or sleep aids.

In still other embodiments, the effective dose of R-(+)-carvedilol, a metabolite of R-(+)-carvedilol, or a salt thereof is at least about 4.5, 5, 5.5, 6, 6,5, 7, 7.5, 8, 8,5, 9, 9.5, 10, 11, 11.5, 12, 12.5, 13, 13.5, 14, 14.5, 15, 15.5, or 16 and up to about 65, 60, 55, 50, 45, 35, 30, 25, or 20 mg/day. The R-(+)-carvedilol, a metabolite of R-(+)-carvedilol, or a salt thereof can be administered once or twice a day, or 1, 2, 3, 4, 5, or 6 times a week, or once every two weeks.

In some embodiments, methods of the invention can comprise administering R-carvedHol at a dose of from about 1.6 mg to about 50 mg daily, optionally about 12.5 mg daily.

In some embodiments, methods of the invention can comprise administering R-carvedilol at a dose of from about 4 mg to about 32 mg twice a day, optionally about 8 mg twice a day.

In some embodiments, methods of the invention can comprise administering R-carvedilol at a dose extrapolated from non-human animal studies using methods known in the art. Optionally, the extrapolation is based on allometric scaling, pharmacokinetically guided approach, minimal anticipated biological effect level, pharmacokinetic-pharmacodynamic modeling, similar drug approach, and microdosing and as described in “Guidance for Industry Estimating the Maximum Safe Starting Dose in Initial Clinical Trials for Therapeutics in Adult Healthy Volunteers” found at Guidance for Industry (fda.gov).

In some embodiments, the dose of R-carvedilol administered is based on the body weight of the subject and includes doses of about 0.1 mg/kg, 0.15mg/kg, 0.2 mg/kg, 0.3 mg/kg, 0.35 mg/kg. 0.4 mg/kg, 0.45 mg/kg or 0.5 mg/kg, optionally the dose is about 0.26 mg/kg.

To gain a better understanding of the invention described herein, the following examples are set forth. It should be understood that these examples are for illustrative purposes only. Therefore, they should not limit the scope of this invention in any way.

### EXAMPLES

### Example 1: Limiting RyR2 Open Time Prevents AD-Associated Intrinsic Hyperexcitability Of Hippocampal CA1 Pyramidal Neurons

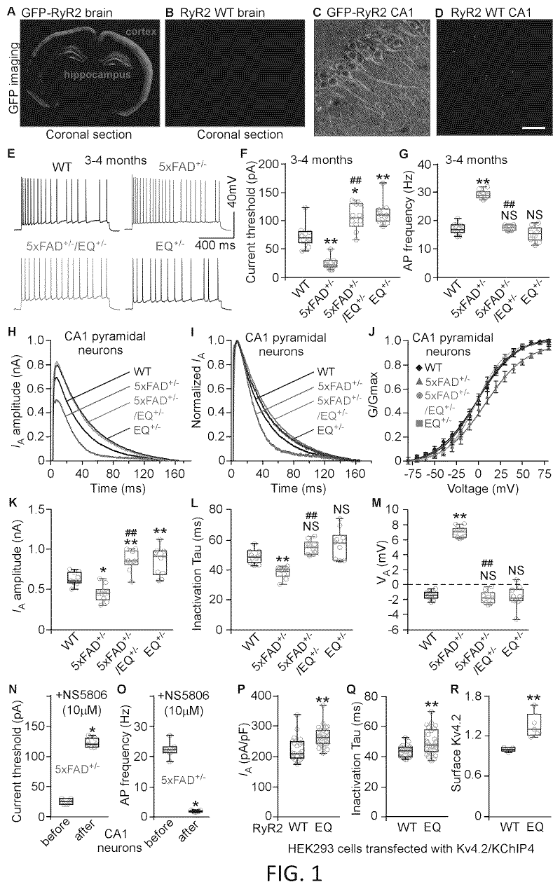

Intrinsic hyperexcitability of hippocampal CA1 pyramidal neurons has been implicated in AD pathogenesis in animal models (Brown et al., 2011; Kerrigan et al., 2014; Scala et al., 2015; Šišková et al., 2014). Thus, it is of interest to determine whether limiting RyR2 open time affects AD-associated intrinsic hyperexcitability of CA1 cells. To this end, heterozygous 5×FAD^{+/-} mice (Oakley et al., 2006) were crossbred with heterozygous RyR2 E4872Q^{+/-} mutant mice (Chen et al., 2014). This breeding generated four genotypes: 5×FAD^{+/-}, 5×FAD^{+/-}/E4872Q^{+/-} (5×FAD^{+/-}/EQ^{+/-}) E4872Q^{+/-} (EQ^{+/-}), and wild type (WT). Of note, RyR2 is predominantly expressed in the hippocampus and cortex, as well as the soma and dendrites of CA1 pyramidal neurons, as revealed by fluorescence imaging of GFP-tagged RyR2 brain sections (Hiess et al., 2015) (FIG. 1A-D).

Whole-cell patch-clamp recordings of 3-4 months old CA1 pyramidal neurons in brain slices to assess intrinsic excitability were performed. In 5×FAD^{+/-} CA1 neurons, the threshold current for inducing action potential (AP) firing was markedly reduced and the frequency of current-induced AP firing was increased compared to WT cells (FIG. 1E-G). Notably, the E4872Q^{+/-} mutation substantially increased the threshold current for AP firing and prevented the increased frequency of AP firing in 5×FAD^{+/-}/EQ^{+/-} and EQ^{+/-} CA1 pyramidal neurons (FIG. 1E-G). Thus, limiting RyR2 open time markedly constrains intrinsic excitability of 5×FAD^{+/-}/EQ^{+/-} and EQ^{+/-} CA1 pyramidal neurons.

### Example 2: Limiting RyR2 Open Time Upregulates the A-Type K⁺ Current of CA1 Pyramidal Neurons

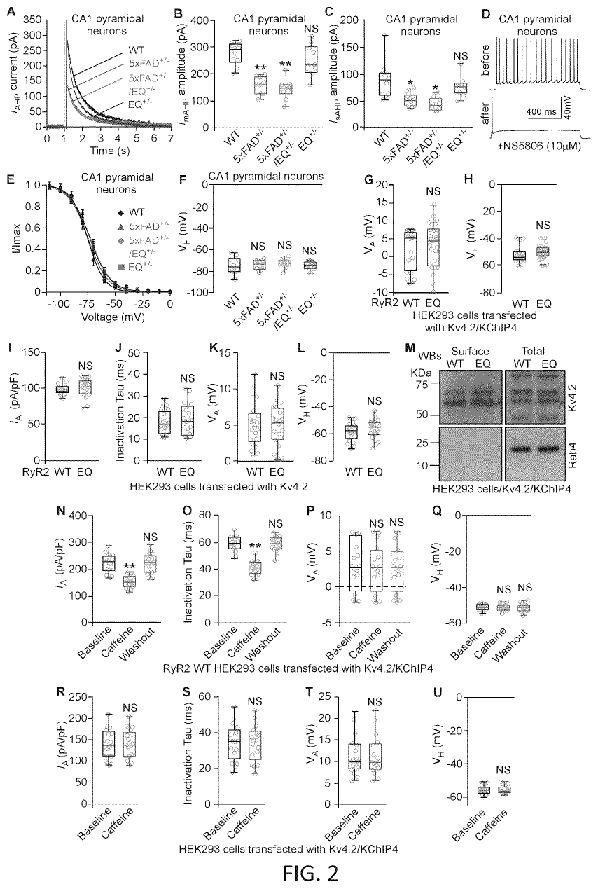

Next the after-hyperpolarization current (I_{AHP}) was measured because I_{AHP} influences intrinsic excitability (Bodhinathan et al., 2010; van de Vrede et al., 2007). In 5×FAD^{++/-} CA1 pyramidal neurons, the medium and slow components of I_{AHP} were substantially reduced compared to WI (FIG. 2A-C) (FIG. 1E-G). There was no significant difference in I_{AHP} between 5×FAD^{+/-} and 5×FAD^{+/-}/EQ^{+/-} and between WT and EQ^{+/-} neurons (FIG. 2A-C). Thus, the E4872Q^{+/-} mutation did not significantly affect I_{AHP} of 5×FAD^{+/-} or WT CA1 pyramidal neurons even though it strongly inhibited intrinsic excitability. Thus, the action of E4872Q^{+/-} in intrinsic excitability is unlikely to be mediated by I_{AHP}.

Hippocampal A-type current downregulation has been implicated in AD-related neuronal hyperactivity (Chen, 2005; Good et al., 1996; Hall et al., 2015; Scala et al., 2015). This led us to assess whether the E4872Q^{+/-} mutation affects the A-type K⁺ current. It was found that the A-type K⁺ current was decreased, decay time (Tau) shortened, and the midpoint for voltage-dependent activation (V_{A}) increased, but the midpoint for voltage-dependent inactivation (V_{H}) was unchanged in 3-4 months old 5×FAD^{+/-} CA1 pyramidal neurons compared to WT (FIG. 1H-M, FIG. 2E, F), consistent with previous reports (Chen, 2005; Good et al., 1996; Hall et al., 2015; Scala et al., 2015). The E4872Q^{+/-} mutation markedly upregulated the A-type K⁺ current of 5×FAD^{+/-}/EQ^{+/-} and EQ^{+/-} hippocampal CA1 neurons compared to WT. Specifically, the E4872Q^{+/-} mutation increased the A-type K⁺ current and decay time without altering V_{A} or V_{H} (FIG. 1H-M, FIG. 2E, F). The A-type K⁺ current agonist, NS5806 (10 μM), dramatically increased the threshold current for inducing AP firing and decreased the frequency of AP firing in 5×FAD^{+/-} CA1 pyramidal neurons (FIG. 1N, O, FIG. 2D). Thus, limiting RyR2 open time upregulates the A-type K⁺ current of CA1 pyramidal neurons.

Hippocampal CA1 neurons express the Kv4.2/KChIP4 channel complex, which is thought to contribute significantly to the A-type K⁺ current (Lin et al., 2010; Rhodes et al., 2004; Serodio and Rudy, 1998; Xiong et al., 2004). KChIP4 is a Ca²⁺ binding protein known to modulate the activity of Kv4.2 (Morohashi et al., 2002). Thus, RyR2-mediated Ca²⁺ release may regulate the A-type K⁺ current by modulating Kv4.2 via KChIP4. To test this, the action of RyR2-E4872Q^{+/-} on the A-type K⁺ current in Kv4.2/KChIP4 transfected HEK293 cells was assessed. The E4872Q^{+/-} mutation increased Kv4.2-mediated current and decay time without altering its voltage-dependent activation or inactivation (FIG. 1P, Q; FIG. 2.1G, H). This action of E4872Q^{+/-} an Kv4.2-mediated current depends on KChIP4, as E4872Q^{+/-} had no effect on Kv4.2-mediated current in HEK293 cells transfected with Kv4.2 alone (i.e., without KChIP4) (FIG. 2 1I-L). The trafficking and surface expression of Kv4.2 are regulated by neuronal activity and Ca²⁺ influx (Kim et al., 2007a). RyR2-mediated Ca²⁺ release may also modulate Kv4.2 trafficking. Indeed, surface labeling experiments revealed that the E4872Q^{+/-} mutation increased the surface expression of Kv4.2 in HEK293 cells (FIG. 1R, FIG. 2. 1M). Caffeine (an agonist of RyR2) decreased Kv4.2-mediated current and decay time without altering its voltage-dependent activation or inactivation (FIG. 2 N-U). Thus, pharmacologically limiting RyR2 open time also affects the A-type K⁺ current.

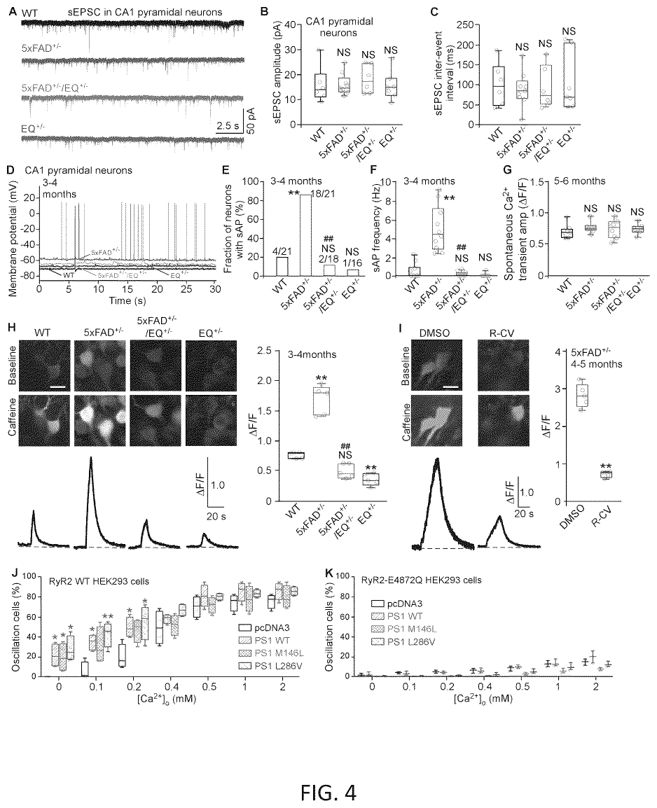

Intracellular Ca²⁺ release through RyRs has been shown to modulate presynaptic activity (Chakroborty et al., 2019; Chakroborty et al., 2012b; Le Magueresse and Cherubini, 2007). Thus, an experiment was performed to determine whether limiting RyR2 open time affects the spontaneous excitatory postsynaptic current (sEPSC) of CA1 pyramidal neurons. There were no significant differences in the amplitude or the inter-event intervals among WT, 5×FAD^{+/-}, 5×FAD^{+/-}/EQ^{+/-}, and EQ^{+/-} CA1 neurons (FIG. 4A-C). This suggests that the action of limiting RyR2 open time in membrane excitability is unlikely to be mediated by changes to synaptic efficacy. Thus, genetically limiting RyR2 open time upregulates the A-type K⁺ current and constrains the intrinsic excitability of CA1 pyramidal neurons.

### Example 3: Limiting RyR2 Open Time Prevents AD-Associated Neuronal Hyperactivity of CA1 Pyramidal Neurons Ex Vivo and In Vivo

To determine whether limiting RyR2 open time can also prevent AD-associated neuronal hyperactivity of CA1 pyramidal neurons, spontaneous neuronal activity (spontaneous AP firing) of CA1 pyramidal neurons in brain slices was measured. Similar to previous reports (Lean et al., 2012; Šišková et al., 2014), the fraction of neurons displaying spontaneous AP firing and the frequency of spontaneous AP firing were markedly increased in 5×FAD^{+/-} CA1 pyramidal neurons compared to WT (FIG. 4D-F). Notably, the E4872Q^{+/-} mutation prevented the enhancement of spontaneous AP firing of 5×FAD^{+/-}/EQ^{+/-} CA1 pyramidal neurons ex vivo in brain slices (FIG. 4D-F).

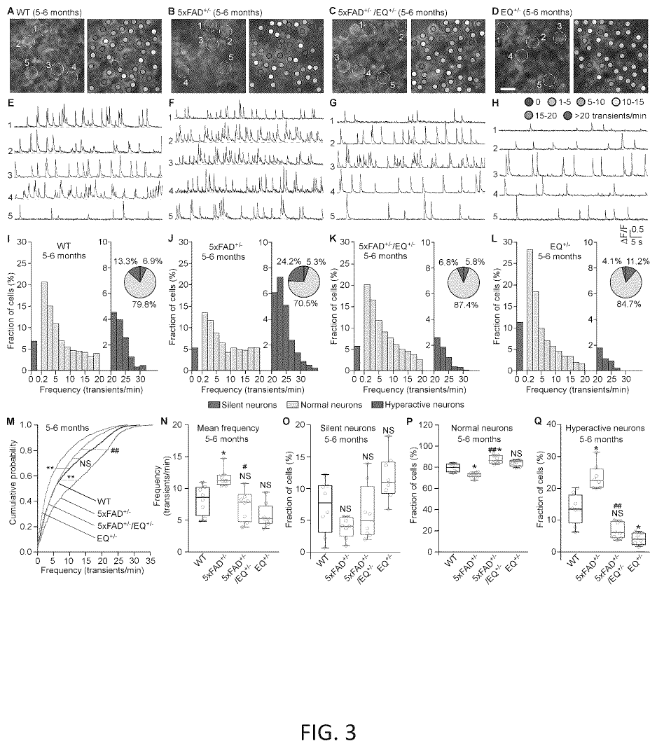

To assess whether limiting RyR2 open time can suppress AD-associated neuronal hyperactivity in vivo, the double heterozygous 5×FAD^{+/-}/E4872Q^{+/-} mice were crossed with the heterozygous Thy-1 GCaMP6f^{+/-} transgenic mice to introduce the GCaMP6f^{+/-} transgene into each of the four genotypes. GCaMP6f is a fast, ultrasensitive Ca²⁺ sensing protein capable of detecting individual action potentials in neurons with high reliability (Chen et al., 2013; Dana et al., 2014; Peron et al., 2015). In vivo two-photon imaging of GCaMP6f-expressing CA1 pyramidal neurons was performed to monitor spontaneous Ca²⁺ transients, which are widely used to assess the spontaneous neuronal activity of cell populations (Busche et al., 2012; Busche et al., 2008; Chen et al., 2013; Dana et al., 2014; Kerr et al., 2005; Peron et al., 2015; Sato et al., 2007; Zott et al., 2019), Anesthetized 5×FAD^{+/-} 5-6 months old mice exhibited neuronal hyperactivity as evidenced by a significant increase in the fraction of hyperactive neurons (as defined by Busche et al. (Busche et al., 2012)) and in the mean frequency of spontaneous Ca²⁺ transients, and a significant decrease in the fraction of normal neurons, compared to WT (FIG. 3A, B, E, F, I, J, N, P, Q). There was no significant difference in the fraction of silent neurons between WT and 5×FAD^{+/-} mice (FIG. The frequency distributions of hyperactive neurons in WT and 5×FAD^{+/-} mice are similar (FIG. 3I, J). This is consistent with previous reports (Busche et al., 2012; Busche et al., 2019; Lerdkrai et al., 2018). Cumulative probability analysis also revealed that 5×FAD^{+/-} mice have markedly increased overall spontaneous neuronal activity compared to WT (P<0.0001) (FIG. 3M).

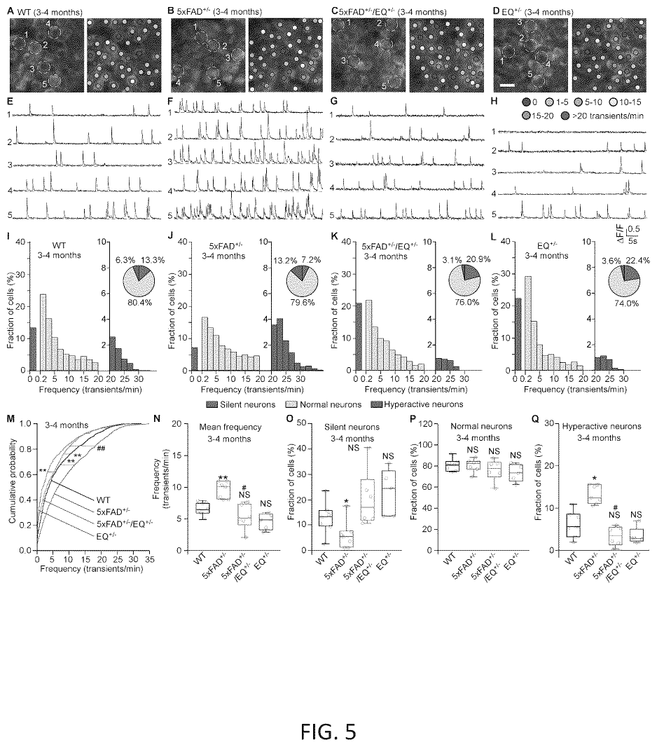

Notably, the RyR2 E4872Q^{+/-} mutation markedly decreased the fraction of hyperactive neurons and the mean frequency of spontaneous Ca²⁺ transients, increased the fraction of normal neurons, and reduced the overall spontaneous neuronal activity (as revealed by the cumulative probability analysis) in 5×FAD^{+/-}EQ^{+/-} and EQ^{+/-} mice, compared to 5×FAD^{+/-} and WT, respectively (FIG. 3). On the other hand, consistent with previous studies (Lerdkrai et al., 2018), there was no significant difference in the amplitude of spontaneous Ca²⁺ transients among different genotypes (FIG. 4G). Neuronal hyperactivity already occurred in 3-4 months old 5×FAD^{+/-} mice as evidenced by a significant increase in the fraction of hyperactive neurons, the mean frequency of spontaneous Ca²⁺ transients, and the overall neuronal activity compared to WT at the same age (FIG. 5). Similarly, the RyR2 E4872Q^{+/-} mutation decreased the fraction of hyperactive neurons and the mean frequency of spontaneous Ca²⁺ transients, and reduced the overall spontaneous neuronal activity in 3-4 months old 5×FAD^{+/-}/EQ^{+/-} mice compared to 5×FAD^{+/-} (FIG. 5). Overall, this shows that limiting RyR2 open time in 5×FAD^{+/-} mice prevents AD-associated neuronal hyperactivity of CA1 pyramidal neurons ex vivo and in vivo.

### Example 4: Limiting RyR2 Open Time Prevents AD-Associated Enhanced RyR2 Function of CA1 Pyramidal Neurons

Two-photon Ca²⁺ imaging of CA1 pyramidal neurons in brain slices prepared from GCaMP6f-expressing WT, 5×FAD^{+/-}, 5×FAD^{+/-}/EQ^{+/-}, and EQ^{+/-} mice was carried out. Consistent with other AD mouse models (Bruno et al., 2012; Chakroborty et al., 2012a; Chakroborty et al., 2009), caffeine induced Ca²⁺ release in 5×FAD^{+/-} CA1 pyramidal neurons was markedly enhanced compared to WT cells (FIG. 4H). On the other hand, caffeine-induced Ca²⁺ release in 5×FAD^{+/-}/EQ^{+/-} or EQ^{+/-} CA1 pyramidal neurons was unchanged or reduced (respectively) compared to WT cells (FIG. 4H). These data indicate that RyR2 function is markedly enhanced in 5×FAD^{+/-} CA1 pyramidal neurons, and this aberrant RyR2 activation is prevented by the RyR2-E4872Q^{+/-} mutation.

The impact of presenilin 1 (PS1) WT and PS1 mutations M146L and L286V on RyR2 function was also assessed by measuring the propensity for spontaneous Ca²⁺ release in RyR2-expressing HEK293 cells transfected with or without PS1 WT or mutants (Chen et al., 2014; Jiang et al., 2005; Jiang et al., 2004). Consistent with early studies (Chan et al., 2000; Rybalchenko et al., 2008; Wu et al., 2013), PS1 WT and mutations markedly enhanced RyR2-mediated spontaneous Ca²⁺ release (FIG. 4J). The E4872Q^{+/-} mutation diminished this spontaneous Ca²⁺ release in all RyR2-expressing cells (FIG. 4K). Thus, limiting RyR2 open time prevents AD-induced aberrant activation of RyR2-mediated Ca²⁺ release.

### Example 5: Limiting RyR2 Open Time Prevents AD-Associated Memory Loss and Hippocampal LTP Deficit

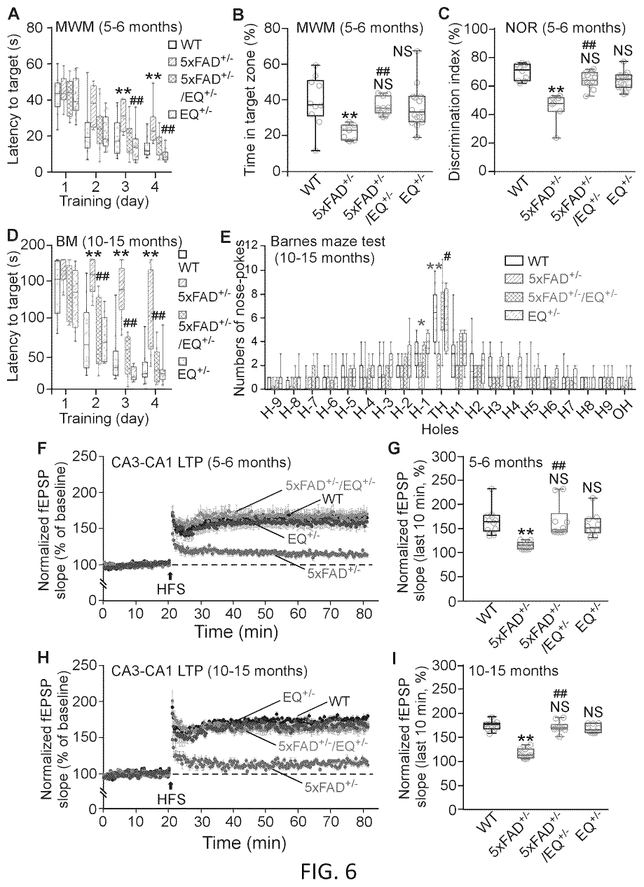

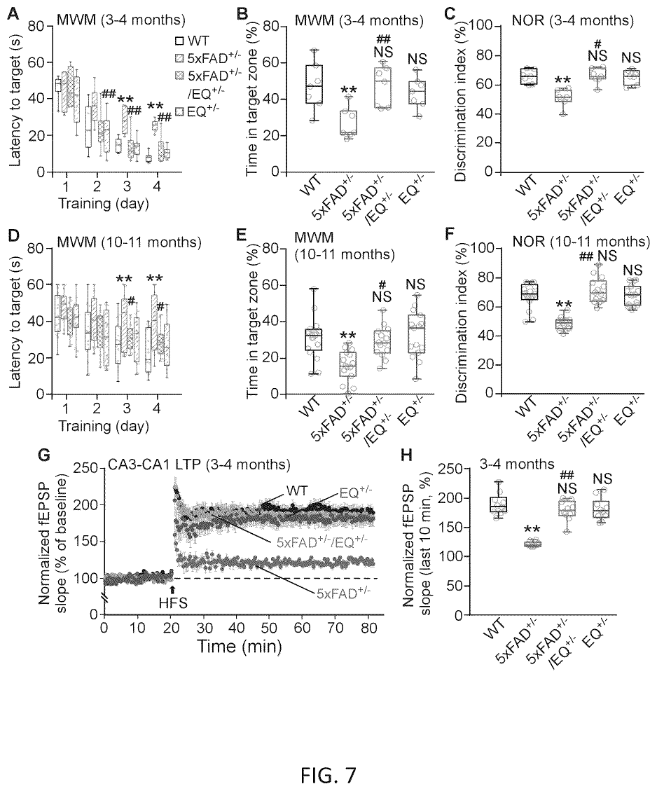

Morris water maze (MWM) and novel object recognition (NOR) tests were performed to assess whether the RyR2 E4872Q^{+/-} mutation prevents the characteristic memory loss in 5×FAD^{30 /-} mice. 5×FAD mice (5-6 months) have significant impairment in learning and memory (FIG. 6A-C) as evidenced by significantly increased latency to target, decreased time spent in the target zone in the MWM, and reduced discrimination index in the NOR test compared to WT. Remarkably, the E4872Q^{+/-} mutation prevented these deficits in 5×FAD^{+/-}/EQ^{+/-} mice. There were no significant differences in MWM or NOR tests between 5-6 months old 5×FAD^{+/-}/EQ^{+/-} and WT or between EQ^{+/-} and WT mice (FIG. 6A-C). Similar MWM and NOR test outcomes were also observed in 3-4 months old mice (FIG. 7A-C). To determine whether the E4872Q^{+/-} mutation could prevent learning and memory deficits in aged 5×FAD^{+/-} mice, Barnes maze (BM) and MWM tests on 10-15 months old mice were performed. As expected, the older 5×FAD^{+/-} mice had severe learning and memory impairments (FIG. 6D, E, FIG. 7D-F). Importantly, the E4872Q^{+/-} mutation prevented these learning and memory impairments in 5×FAD^{+/-}/EQ^{+/-} mice even in this late stage of AD.

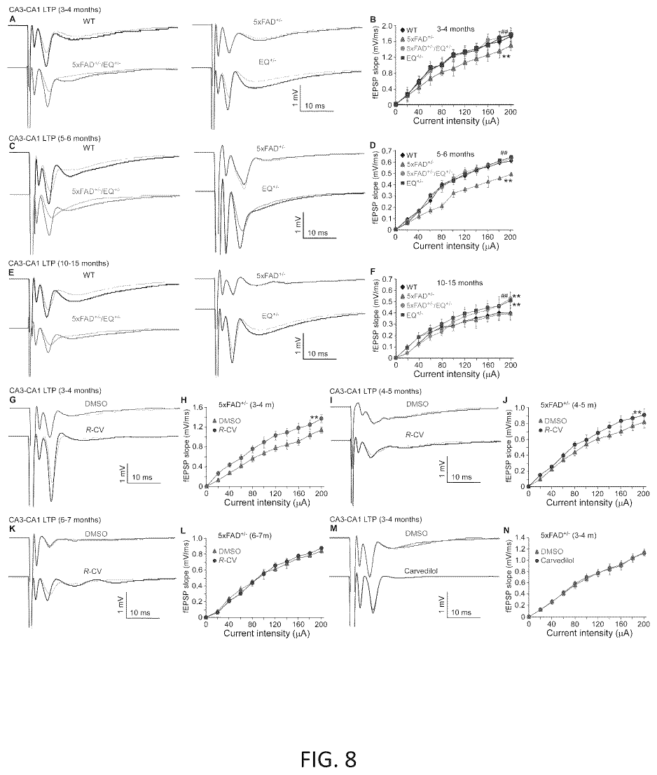

The effect of the E4872Q^{+/-} mutation on learning and memory was also assessed at the cellular level by measuring hippocampal LTP in brain slices. Consistent with our behavioral studies, 5×FAD^{+/-} mice at 3-4, 5-6, 10-15 months of age showed little or no hippocampal LTP (FIG. 6F-I; FIG. 7G, H). The E4872Q^{+/-} mutation prevented these hippocampal LTP impairments as evidenced by the similar levels of hippocampal LTP in the WT, 5×FAD^{+/-}/EQ^{+/-}, and EQ^{+/-} mice (FIG. 6F-I; FIG. 7G, H). 5×FAD^{+/-} mice also displayed slightly reduced hippocampal basal synaptic transmission as revealed by the reduced fEPSP slope in relation to the current input (FIG. 8A-F), similar to a previous report (Waring et al., 2012). Thus, limiting RyR2 open time prevents hippocampal LTP impairment and memory loss in 5×FAD^{+/-} mice.

### Example 6: R-Carvedilol Prevents and Rescues Neuronal Hyperactivity and Memory Loss in 5×FAD^{+/-} Mice

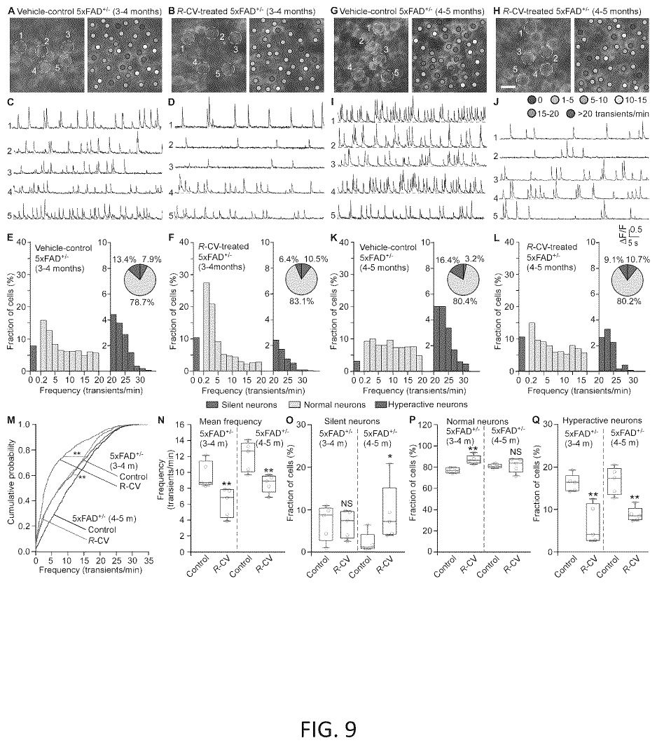

It was previously shown two studies that R-carvedilol shortened RyR2 open time in cardiac cells (Zhang et al., 2015; Zhou et al., 2011). Given the effect of the E4872Q^{+/-} mutation on hippocampal neuronal activity, this agent was explored to assess its effect on neuronal RyR2 open time, neuronal hyperactivity, and AD progression. To test this, 2-3 months old 5×FAD^{+/-} mice (i.e. before the occurrence of AD pathology) or 3-4 months old 5×FAD^{+/-} mice (i.e. after the occurrence of AD pathology) (Oakley et al., 2006) were pre-treated with R-carvedilol (3.2 mg/kg/day) or its vehicle control (DMSO) for one month. The R-carvedilol pre-treatment prevented and rescued neuronal hyperactivity of 5×FAD^{+/-} hippocampal CA1 neurons in vivo as evidenced by the observation that the fraction of hyperactive neurons, the mean frequency of spontaneous Ca²⁺ transients, and the overall spontaneous neuronal activity were significantly lower in R-carvedilol pre-treated 5×FAD^{+/-} mice than those in vehicle pre-treated mice at both ages (FIG. 9), but similar to those in WT (FIG. 5). Caffeine-induced Ca²⁺ release was also markedly reduced in R-carvedilol pre-treated 5×FAD^{+/-} CA1 pyramidal neurons compared to DMSO-treated (control) cells in brain slices (FIG. 4I). Thus, R-carvedilol, like the RyR2-E4872Q^{+/-} mutation, can reduce neuronal hyperactivity and prevent AD-induced aberrant activation of RyR2-mediated Ca²⁺ release.

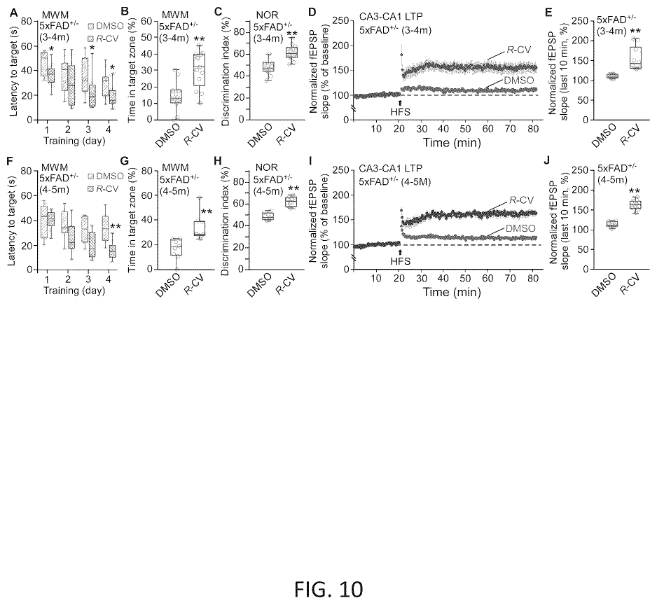

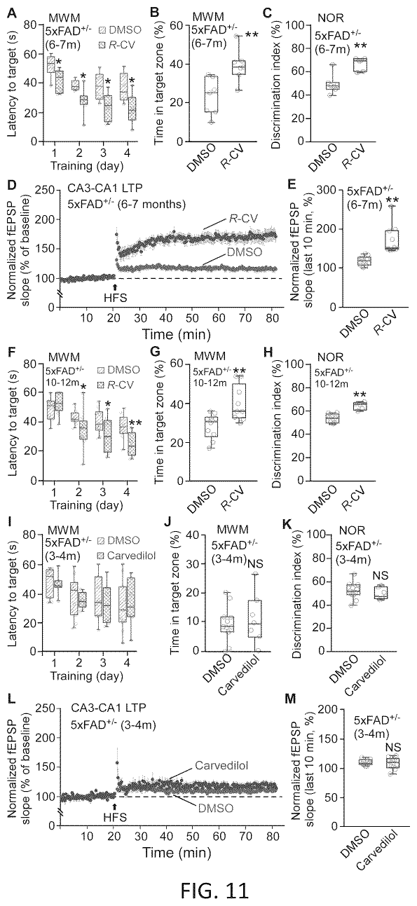

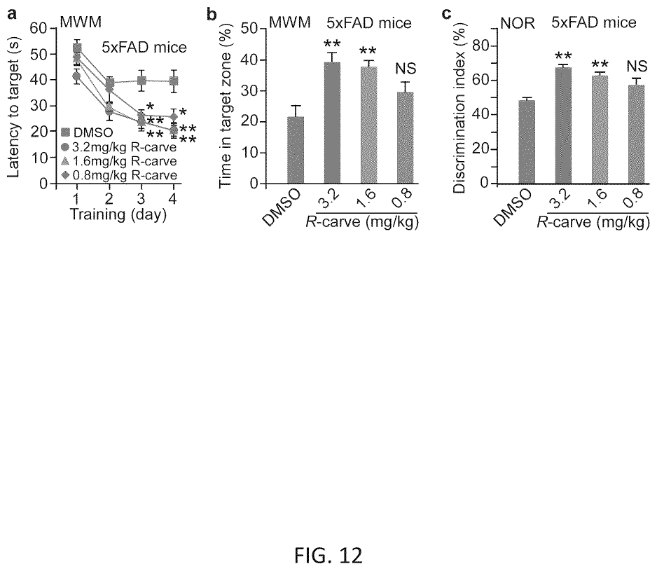

R-carvedilol pre-treatment of 2-3 months old 5×FAD^{+/-} mice (before AD symptoms) also prevented memory loss and LTP impairments (FIG. 10A-E), and R-carvedilol pre-treatment of 3-4 months old 5×FAD^{+/-} mice (after AD symptom) rescued these defects (FIG. 10F-J). To further assess whether R-carvedilol pre-treatment is still effective in late stages of AD, the drug was tested on 6-7 and 10-12 months old 5×FAD^{+/-} mice. R-carvedilol pre-treatment rescued memory deficits even in 6-7 and 10-12 months old 5×FAD^{+/-} mice (i.e. late stages of AD) (FIG. 11A-H). The reduced basal synaptic transmission in 5×FAD^{+/-} hippocampal brain slices was also restored by R-carvedilol pre-treatment (FIG. 8G-L). The clinically-used carvedilol racemic mixture (3.2 mg/kg/day) was also tested. Racemic carvedilol did not prevent learning and memory deficits or LTP impairment in 3-4 months old 5×FAD^{+/-} mice (FIG. 8M, N; FIG. 11I-M). Furthermore, the beneficial effect of R-carvedilol on learning and memory was dose-dependent. R-carvedilol at a dose of 3.2 mg/kg/day or 1.6 mg/kg/day, but not at 0.8 mg/kg/day, rescued learning and memory impairments in 5×FAD mice (FIG. 12).

### Example 7: Limiting RyR2 Open Time Does not Affect AβAccumulation but Protects Against Neuronal Cell Death and Dendritic Spine Loss

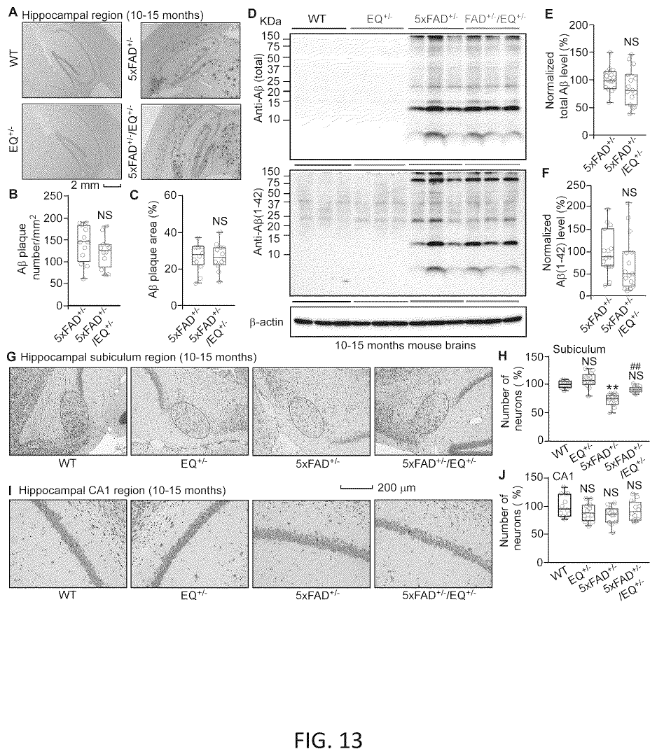

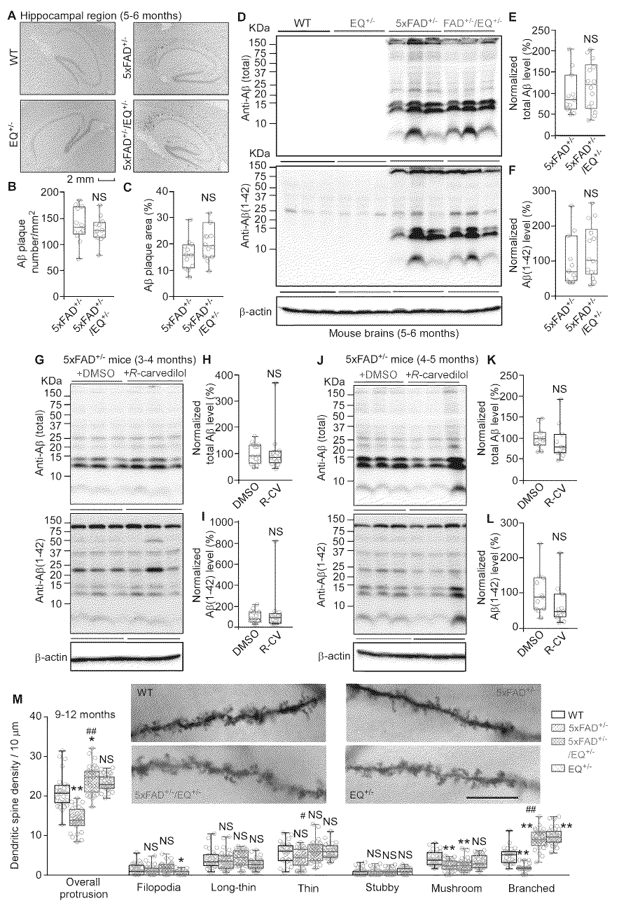

Immunohistochemical staining and immunoblotting analyses were performed to assess the effect of the RyR2-E4872Q^{+/-} mutation on Aβ accumulation. There was no significant difference in the number or the area of Aβ plaques detected in the hippocampus of 10-15 months old 5×FAD^{+/-} and 5×FAD^{+/-}/EQ^{+/-} mice (FIG. 13A-C). Immunoblotting analyses also showed no significant difference in the total Aβ or Aβ (1-42) level in 10-15 months old and 5×FAD^{+/-}/EQ^{+/-} brain tissue homogenates (FIG. 13D-F). No Aβ plaques were detected in WT or or EQ^{+/-} brain slices, and no specific Aβ signals were detected in WT or EQ^{+/-} brain tissue homogenates. Similar results were also observed in 5-6 months old mice (FIG. 14A-F). Thus, limiting RyR2 open time does not significantly alter Aβ accumulation.

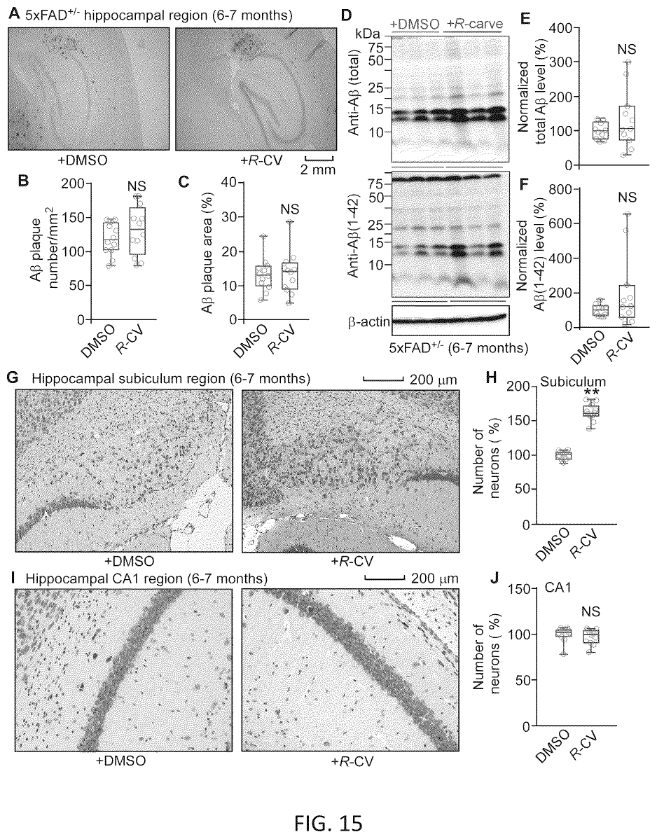

The RyR2 E4872Q^{+/-} mutation influence on neuronal cell death was assessed. Consistent with previous reports (Jawhar et al., 2012; Oakley et al., 2006), the number of pyramidal neurons in the subiculum (but not CA1) region was significantly reduced in 10-15 months old 5×FAD^{+/-} brain slices (FIG. 13G-J). The E4872Q^{+/-} mutation prevented this subiculum neuronal cell loss as evidenced by the similar number of subiculum pyramidal neurons in 5×FAD^{+/-}/EQ^{+/-} and WT brains (FIG. 13H). Like the E4872Q^{+/-} mutation, R-carvedilol pre-treatment also protected against subiculum neuronal cell loss without affecting Aβ accumulation (FIG. 15; FIG. 14G-L).

Golgi staining was performed to determine whether the RyR2 E4872Q^{+/-} mutation affects spine density and morphology of 5×FAD^{+/-} CA1 pyramidal neurons. Consistent with previous studies (de Pins et al., 2019; Kim et al., 2020; Yang et al., 2018), the density of overall protrusions, and specifically, the density of mushroom and branched spines, was significantly reduced in the 5×FAD^{+/-} CA1 pyramidal neurons compared to WI cells (FIG. 14M). The E4872Q^{+/-} mutation (limiting RyR2 open time) prevented the loss of overall protrusions and branched spines, but not the loss of mushroom spines, of 5×FAD^{+/-}/EQ^{+/-} CA1 pyramidal cells (FIG. 14M). The E4872Q^{+/-} mutation also decreased the density of filopodia spines and increased the density of branched spines of the EQ^{+/- CA}1 cells compared to WT cells (FIG. 14M). This indicates that limiting RyR2 open time prevents dendritic spine loss in 5×FAD^{+/-} mice despite extensive Aβ accumulation.

### Example 8: Limiting RyR2 Open Time Prevents Learning and Memory Impairments in the 3×TG Alzheimer's Disease Mouse Model

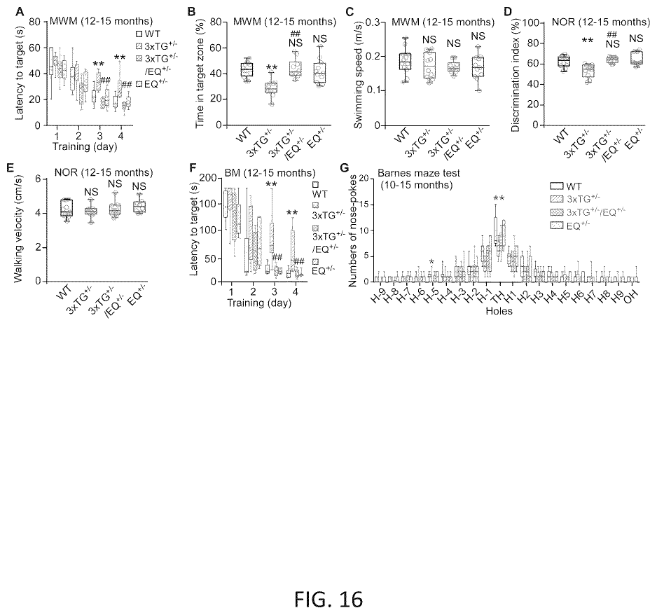

To assess whether limiting RyR2 open time can also prevent AD-related learning and memory impairments in a relatively slow, late occurring AD mouse model, 3×TG, heterozygous RyR2-E4872Q^{+/-} mutant mice were crossbred with the 3×TG^{+/-} AD mouse model. This breeding generated four genotypes: 3×TG^{+/-}, 3×TG^{+/-}/E4872Q^{+/-}(3×TG^{+/-}/EQ^{+/-}), E872Q^{+/-}(EQ^{+/-}) and wild type (WT). Morris water maze (MWM) and novel object recognition (NOR) tests were performed on the 3×TG^{+/-}, 3×TG^{+/-}/EQ^{+/-}, EQ^{+/-} and WT mice at the age of 12-15 months old. As shown in FIG. 16A, 3×TG^{+/-} mice displayed significantly increased latency to find the hidden platform and significantly reduced the time spent in the target zone during the probing test on day 5 of MWM test (FIG. 16B) compared to WT mice, which are consistent with those reported previously (Baeta-Corral & Gimenez-Llort, 2015; Billings, Oddo, Green, McGaugh, & LaFerla, 2005; D. Kim, Cho, & Kang, 2019). There were no significant differences in the swimming speed during the MWM test among different genotypes (FIG. 16C). The 3×TG^{+/-} mice also displayed a significantly reduced discrimination index in the NOR test compared to WT (FIG. 16D). There were no significant differences in the walking velocity during the NOR test among different genotypes (FIG. 16E). Notably, the RyR2-E4872Q^{+/-} mutation prevented these learning and memory impairments in 3×TG^{+/-} mice as evidenced by the observation that there were no significant differences in MWM or NOR tests between 3×TG^{+/-}/EQ^{+/-} and WT mice at the age of 12-15 months old. There were also no significant differences in MWM or NOR tests between EQ^{+/-} and WT mice (FIG. 16A-E).

The MWM test could be stressful to mice and may potentially affect their behaviors (Holscher, 1999; J. J. Kim, Lee, Han, & Packard, 2001). Thus, the relatively less stressful test, Barnes maze (BM), was also performed on 3×TG^{+/-}, 3×TG^{+/-}/EQ^{+/-}, EQ^{+/-} and WT mice at the age of 12-15 months old, Consistent with those observed in the MWM test, 3×TG^{+/-} mice exhibited impaired learning and memory as evidenced by the increased latency to find the target hole and the reduced number of nose-pokes on the target hole (FIG. 16F, G). Furthermore, the E4872Q^{+/-} mutation prevented these learning and memory impairments in the 3×TG^{+/-} mice as evidenced by the data that there were no significant differences in the BM test between 3×TG^{+/-}/EQ^{+/-} and WT mice (FIG. 16F, G). There were also no significant differences in the BM tests between EQ^{+/-} and WT mice (FIG. 16F, G). Collectively, these behavioral tests indicate that limiting RyR2 open time prevents hippocampal learning and memory impairments in the relatively slow, late occurring 3×TG AD mouse model (compared to 5×FAD mice).

### Example 9: Limiting RyR2 Open Time Prevents Hippocampal LIP Deficit in 3×TG Mice

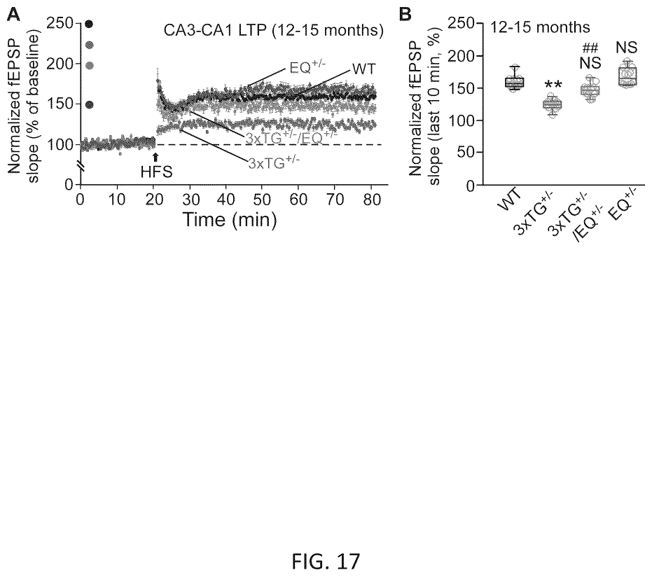

Long-term potentiation (LTP) deficiency is a well-known neuronal dysfunction in 3×TG^{+/-} mice (Chakroborty et al., 2009; Clark et al., 2015; Oddo et al., 2003). To determine whether limiting RyR2 open time could also prevent LTP deficit in the 3×TG^{+/-} mice, field excitatory postsynaptic potential (fEPSP) recordings were performed at the Schaffer collateral region in hippocampal slices from 12-15 months old 3×TG^{+/-}, 3×TG^{+/-}/EQ^{+/-}, EQ^{+/-} and WT mice. Consistent with our behavioral studies, 3×TG^{+/-} mice showed reduced LTP (i.e. a decreased level of potentiation of the fEPSP slope after a high frequency stimulation, HFS) compared to WT (FIG. 17). Notably, the E4872Q^{+/-} mutation prevented this LTP deficit in the 3×TG^{+/-} mice as evidenced by a similar LTP level observed in 3×TG^{+/-}/EQ^{+/-} and WT mice. There was also no significant difference in LIP between EQ^{+/-} and WT mice (FIG. 17). Thus, these data show that limiting RyR2 open time prevents hippocampal LTP deficit in 3×TG^{+/-} mice.

### Example 10: Limiting RyR2 Open Time Prevents Dendritic Spine Loss of 3×TG CA1 Pyramidal Neurons

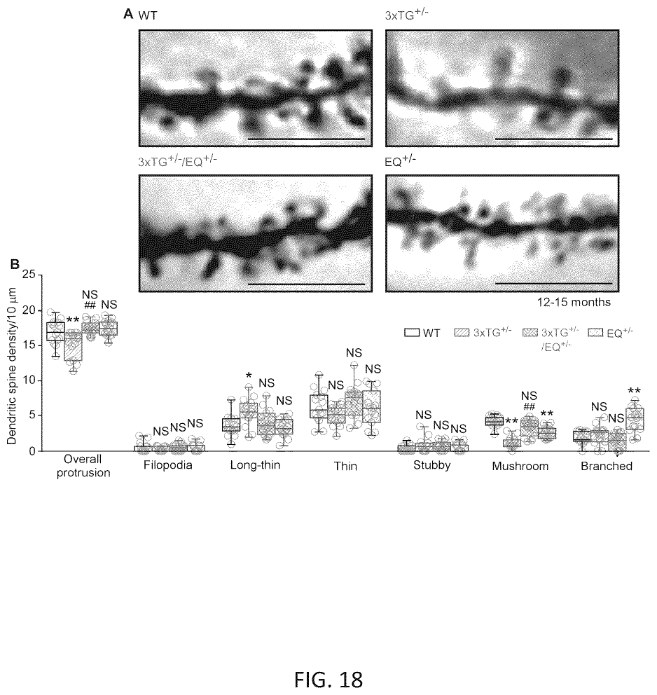

To determine the impact of limiting RyR2 open time on AD-related changes in spine structure, Golgi staining was used to assess the spine density and morphology of CA1 pyramidal neurons in 12-15 months old 3×TG^{+/-}, 3×TG^{+/-}/EQ^{+/-}, EQ^{+/-} and WT mice. To be able to trace the fine structures of spines along a relatively long dendrite, a series of Z-stack images of the Golgi-stained apical dendrites of CA1 neurons using a 100× objective were taken. These Z-stack images were then used to reconstruct the three-dimensional (3D) dendritic segments using ImageJ and the RECONSTRUCT program (Risher et al., 2014). Different types of spine, including filopodia, long thin, thin, stubby, mushroom and branched spines, as defined previously (Risher et al., 2014), could be clearly identified from the reconstructed 3D dendritic segments. Note that the representative images of dendritic spines shown in FIG. 18 were 2D images at a single Z-(focal) plane, in which some of the spines were out of focus. By carefully tracing and analyzing the 3D reconstructed dendritic segments, it was found that, consistent with previous studies (Baglietto-Vargas et al., 2018; Bittner et al., 2010; Pedrazzoli et al., 2019), the overall density of spine protrusions in hippocampal CA1 pyramidal neuron apical dendrites of 3×TG^{+/-} mice was significantly reduced compared to that in WT. Furthermore, spine subtype analyses revealed that the reduction in overall spine protrusions in the 3×TG^{+/-} CA1 pyramidal neurons resulted mainly from a reduced density of the mushroom type of spines. Also note that the number of mushroom spines in WT CA1 neurons was similar to that reported previously (Bevan et al., 2020; de Pins et al., 2019; Ryskamp et al., 2019). Remarkably, the E4872Q^{+/-} mutation prevented the loss of overall protrusions and the mushroom type of spines in the 3×TG^{+/-}/EQ^{+/-} mice (FIG. 18). These data indicate that limiting RyR2 open time prevents hippocampal CA1 dendritic spine loss in 3×TG^{+/-} mice.

### Example 11: Limiting RyR2 Open Time Prevents Neuron Loss in the Subiculum Region of 3×TG Mice

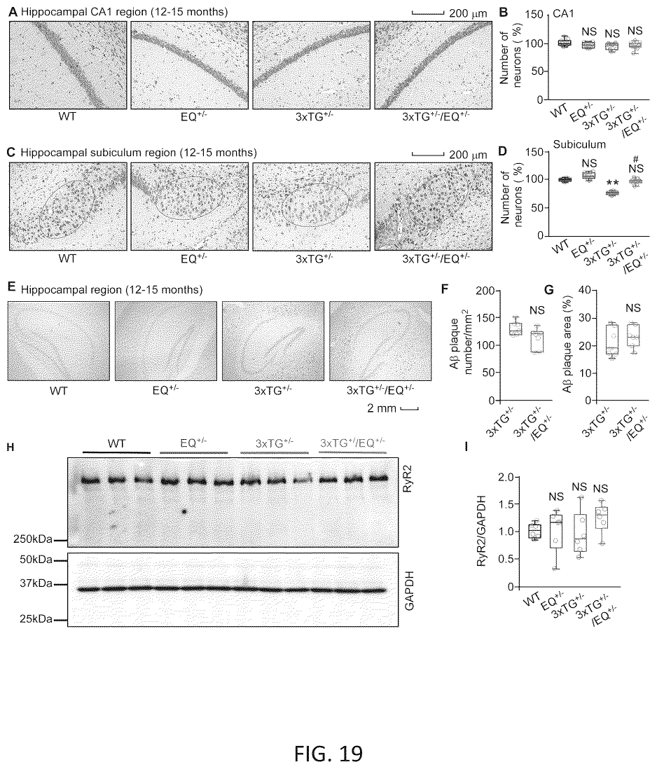

To test whether the number of neurons in the subiculum region of the 3×TG mice was reduced, Nissl staining of hippocampal brain slices from 12-15 months old 3×TG^{+/-}, 3×TG^{+/-}/EQ^{+/-}, EQ^{+/-} and WT mice was performed. To minimize potential regional differences, only hippocampal sagittal sections 20-50 μm from the mid-line for each genotype were analyzed. To facilitate the comparison of neuron numbers in the subiculum region in different hippocampal slices from different genotypes, the number of neurons within an area of the same size that is large enough to cover 70-90% of the subiculum region in all hippocampal slices from all genotypes (note that the size of the visible subiculum area varies from slice to slice) was counted. Similar to those observed in 5×FAD mice (Jawhar et al., 2012; Oakley et al., 2006; Yao et al., 2020), no significant differences in the number of neurons in the CA1 region in 3×TG^{+/-} hippocampal slices compared to that in WT (FIG. 19A, B) was detected. However, there was a significant neuron loss in the subiculum region in the 3×TG^{+/-} hippocampal slices compared to WT (FIG. 19C, D). Notably, the E4872Q^{+/-} mutation prevented neuron loss in this region in the 3×TG^{+/-} mouse hippocampal slices as evidenced by the similar number of neurons in 3×TG^{+/-}/EQ^{+/-} and WT brain slices (FIG. 19C, D). Therefore, these results show that limiting RyR2 open time prevents neuron loss in 3×TG^{+/-} mice. To assess the effect of the RyR2-E48720Q^{+/-} mutation on Aβ accumulation in the 3×TG^{+/-} mice immunohistochemical staining was performed. There was no significant difference in the number or the area of Aβ plaques in the hippocampal region of 12-15 months old 3×TG^{+/-} and 3×TG^{+/-}/EQ^{+/-} mice (FIG. 19E-G). Note that the expressions of RyR2 in different genotypes were comparable (FIG. 19H, I). Thus, limiting RyR2 open time does not significantly alter Aβ accumulation.

### Example 12: R-Carvedilol Rescues Memory Impairment, LTP Deficit, and Neuron Loss in 3×TG Mice

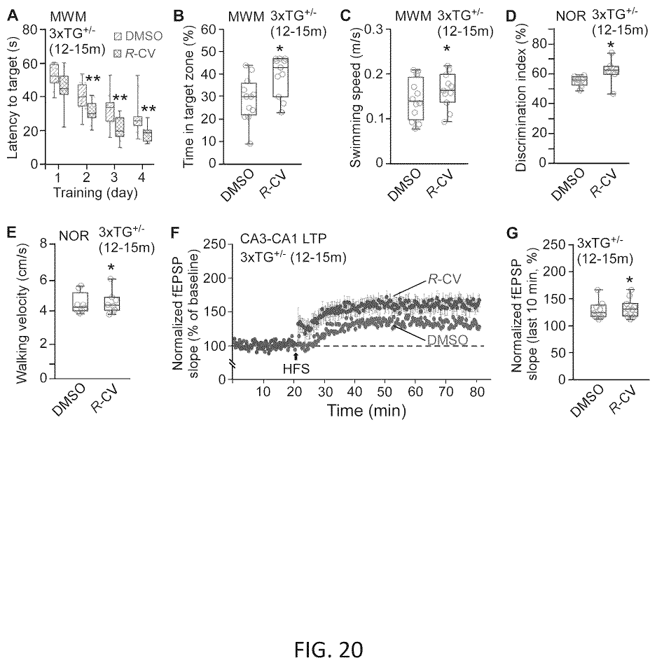

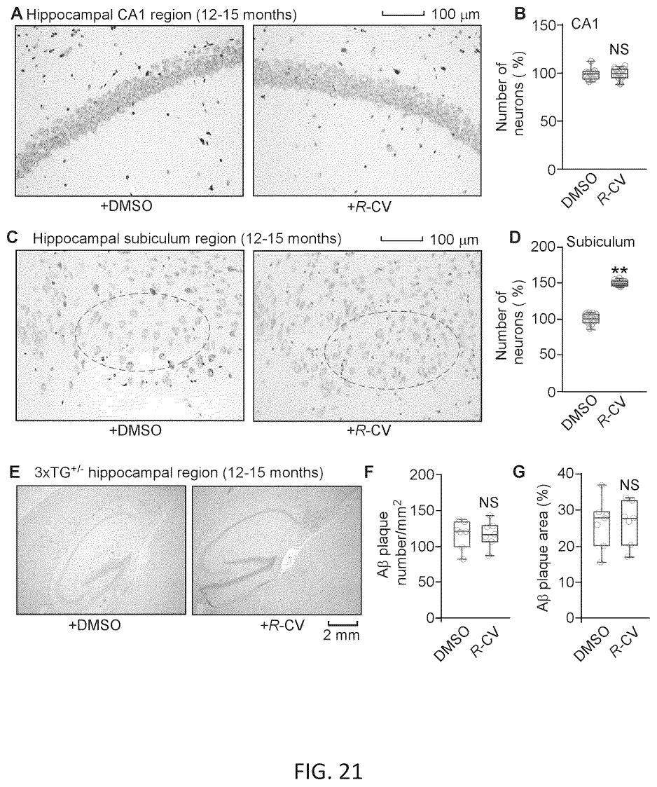

To address whether R-carvedilol can also rescue AD-related deficits in the relatively slow, late occurring AD mouse model, 3×TG, 12-15 months old 3×TG^{+/-} mice were pretreated with R-carvedilol (3.2 mg/kg/day) or DMSO (vehicle control) for one month and conducted behavioral tests, LTP measurements, and histochemical staining. R-carvedilol pre-treatment significantly shortened the latency to the target platform and increased the time spent in the target zone and the swimming speed in the MWM test compared to the DMSO pretreated 3×TG^{+/-} mice (FIG. 20A, B, C). R-carvedilol pre-treatment also increased the discrimination index and the walking velocity in the NOR test in the 3×TG^{+/-} mice compared to the DMSO-pretreated 3×TG^{+/-} mice (FIG. 20D, E). LTP recordings revealed that R-carvedilol pre-treatment significantly enhanced hippocampal LTP in brain slices from 3×TG^{+/-} mice as evidenced by the significantly increased potentiation of fEPSP slope compared to the DMSO pretreated group (FIG. 20F, G). Furthermore, Nissl staining showed that there was no significant difference in the density of neurons in the hippocampal CA1 region between the R-carvedilol- and DMSO-pretreated 3×TG^{+/-} mice (FIG. 21A, B). However, the density of neurons in the subiculum area in the R-carvedilol pretreated 3×TG^{+/-} mice was significantly higher than that in the DMSO-pretreated mice, but it was similar to that in WT (FIG. 21C, D). Immunohistochemical staining was also performed to assess the effect of R-carvedilol on Aβ accumulation in the 3×TG^{+/-} mice. There was no significant difference in the number or the area of Aβ plaques in the hippocampal region of 12-15 months old 3×TG^{+/-} mice between R-carvedilol pre-treatment and DMSO pre-treatment (FIG. 21E, F, G). Taken together, these data show that R-carvedilol rescues memory impairment, LTP deficit, and neuron loss, but not Aβ accumulation in 3×TG^{+/-} mice.

### Methodology of Examples

### Mouse Models

5×FAD mouse model is a rapid, early onset mouse model of Alzheimer's Diseases that has the hallmarks of Alzheimer's Disease in humans. The 5×FAD mouse model displays AD-related neuronal dysfunctions and pathologies as early as 2-3 months (rather than 12 months or longer) (Oakley et al., 2006). The 5×FAD mouse model rapidly develops AD symptoms due to the presence of 5 human familial AD (FAD) mutations, which is different from the slow progression of AD that occurs in the majority of human cases (Jankowsky & Zheng, 2017; Lee & Han, 2013).

The 3×TG AD mouse model of Alzheimer's Disease has a relatively slow progression of disease with late occurring AD symptoms compared to 5×FAD mouse model (Jankowsky & Zheng, 2017; Oddo et al., 2003).

### 5×FAD^{+/31} Mice

Adult genetically engineered mice, 5×FAD^{+/-} (Oakley et al., 2006), RyR2-E4872Q^{+/-} (EQ^{+/-}) (Chen et al., 2014), 5×FAD^{+/-}/RyR2-E4872Q^{+/-} (5×FAD^{+/-}/EQ^{+/-}), and wildtype (WT) littermates of both sexes were used. R-carvedilol (R-CV) (at doses of 0.8, 1.6 or 3.2 mg/kg/day), racemic carvedilol (3.2 mg/kg/day), and vehicle control (DMSO) were delivered to 5×FAD^{+/-} mice in drinking water for one month, starting at different ages before (2-3 months old) or after (3-4 months old) the occurrence of AD pathologies (Oakley et al., 2006). To assess the effects of genetically and pharmacologically limiting RyR2 open time on the prevention and rescue of AD deficits in different stages (early, moderate, and late) of AD progression, animals at different ages (from 2-15 months) were used. As reported (Oakley et al., 2006), there was an age-dependence of AD progression in the 5×FAD^{+/-} mice. No sex-dependent differences in AD progression in these mice was observed. For 2-photon Ca²⁺ imaging experiments, 5×FAD, RyR2 WT, and RyR2 mutant mice were cross-bred with the heterozygous Thy1-GCaMP6f transgenic mice (Chen et al., 2012; Chen et al., 2013) (GP5.17, JAX 025393) to express the GCaMP6f Ca²⁺ sensing probe (driven by the Thy1 promotor) in hippocampal neurons in each of the genotypes used.

### 3×TG^{+/-} Mice

Genetically engineered mice of 12-15 months old, 3×TG+/− (Oddo et al., 2003), RyR2-E4872Q+/− (EQ+/−)(Chen et al., 2014), 3×TG+/−/RyR2-E48720+/− (3×TG+/−/EQ+/−), and WT littermates of both sexes were used. Sex differences, which may produce biological variables, were not investigated in this study. R-carvedilol (R-CV) (3.2 mg/kg/day) and vehicle control (DMSO) were delivered to 3×TG+/− mice in drinking water for one month. As shown previously, only heterozygous RyR2-E4872Q+/− mutant mice were produced as homozygous E4872Q+/+mutation is embryonic lethal (Chen et al., 2014). Functional RyR2s are tetrameric channels formed by 4 RyR2 monomers. The heterozygous E4872Q+/− mutant mice (harboring one WT allele and one E4872Q mutant allele) will produce a mixture of homo- and hetero-tetrameric channels that contain the WT, E48720 mutant, or both WT/E4872Q mutant monomers. Thus, the RyR2-E4872Q mutation can exert its negative impact not only on the function of the E4872Q homo-tetramers, but also on the function of the WT monomer in the WT/E4872Q hetero-tetrameric channels.

### Cell Lines

The Flp-In T-REx HEK293 cell line was obtained from Invitrogen. HEK293 cell lines expressing RyR2 WT or the RyR2 E4872Q mutation were generated using the Flp-In T-Rex HEK293 cell line. HEK293 cell lines were cultured in Dulbecco's Modified Eagle Medium (GIBCO) supplemented with 10% fetal bovine serum (GIBCO), 100 units/ml penicillin and 100 μg/ml streptomycin (GIBCO), 4 mM L-glutamin (GIBCO) and 0.1 mM MEM Non-Essential Amino Acids Solution (GIBCO). All cell lines were cultivated in a humidified incubator with 5% CO₂ at 37° C. and were tested negative for mycoplasma contamination.

### The Synthesis of (R)-Carvedilol

Commercially available (S)-glycidol was converted into its o-nitrobenzenesulfonate (nosylate) by the method of Shiratsuchi et al. The nosylate (9.60 g, 37.0 mmol) in 35 mL of DMF was added dropwise to a cooled (0° C.) solution of 4-hydroxycarbazole (6.90 g, 37.7 mmol) and sodium hydroxide (1.55 g, 38.7 mmol) in 100 mL of DMF and 1 mL of water. Stirring was continued for 5 h at 0° C. and then at room temperature overnight. The mixture was diluted with brine, extracted with ethyl acetate and the combined organic layers were washed with saturated aqueous sodium bicarbonate, 1 N sodium hydroxide and brine. The resulting solution was dried over anhydrous sodium sulfate, concentrated under vacuum and subjected to flash chromatography over silica-gel (elution with 2%-4% ethyl acetate—toluene) to afford 7.95 g (90%) of the corresponding 4-[(R)-1-oxiranylmethoxy]-9H-carbazole as a white solid, mp 159-160° C., with ¹H and ¹³C NMR spectra identical to those of the racemic material.

2-(2-Methoxyphenoxy)ethylamine (7.00 g, 41.9 mmol) in 15 mL of isopropanol was added dropwise to the above product (5.49 g, 22.9 mmol) in 35 mL of isopropanol. The mixture was refluxed for 1.5 h. The solvent was evaporated and the product was purified by flash chromatography over silica-gel (elution with 3%-7% of methanol-dichloromethane) to provide 6.20 g (67%) of (R)-(+)-carvedilol as a white solid foam, mp 115-116° C.; [α]D²¹+17.3° (c 1.0, acetic acid); lit, mp 121-123° C.; [α]_{D}²⁰+18.4° (c 1, acetic acid). Elemental analysis calculated for C₂₄H₂₆N₂O₄: C 70.93, H 6.45, N 6.89; found: C 70.75, H 6.67, N 6.85. The product gave IR, ¹H and ¹³C NMR spectra identical to those of authentic racemic carvedilol.

### Method Details

### Acute Slices Preparation

Acute brain slices were prepared according to the published procedures with some modifications (Ting et al., 2014; Ting et al., 2018).

### Whole-Cell Patch-Clamp Recordings

For whole-cell patch-clamp recordings, slices were transferred to a submerged recording chamber perfused with carbogenated external solutions at a flow rate of 4-6 mL/min at room temperature. Action potentials (APs) were measured in hippocampal CA1 pyramidal neurons in transverse hippocampal slices (260 μm) from all genotypes of 3-4 months old mice using whole-cell patch-clamp with an Axopatch 700B amplifier (Axon Instruments). Hippocampal CA1 pyramidal neurons as they play a critical role in neuronal activity, learning and memory (Brager and Johnston, 2007; Kerrigan et al., 2014; Tamagnini et al., 2015; Xu et al., 2005). Membrane potentials in 5-6 months old or older neurons were not measured as aged neurons are difficult to patch. AP firing was recorded in an external solution (NaCl, 124 mM; KCl, 2.5 mM; NaH₂PO₄, 1.25 mM; NaHCO₃, 24 mM; HEPES, 5 mM; glucose, 12.5 mM; MgCl₂, 2 mM; and CaCl₂, 2 mM; pH 7.4 adjusted with NaOH) and soft-glass recording pipettes (Sutter Instruments; Novato CA) filled with an internal solution (potassium gluconate, 135 mM, KCl, 10 mM, HEPES, 10 mM, CaCl₂, 1 mM, MgCl₂, 1 mM, EGTA, 10 mM, ATP, 1 mM, GTP, 0.1 mM, and pH 7.3 adjusted with KOH). The pipette resistance was 4-6 MΩ after filling with internal solution. For spontaneous AP recording, cells were hold at −70 mV and recorded for 3 min. For the measurement of current-injection triggered APs, 0.05 mM 2-amino-5-phosphonovaleric acid (APV), 0.02 mM 6,7-dinitroquinoxaline-2,3-dione (DNQX) and 0.1 mM picrotoxin were added to the external solution to block synaptic activity. APs were initiated by injecting current from 0 to 300 pA for 1 s in 10 pA steps at 10 s intervals. For testing the Kv4 channel agonist NS5806, APs were measured before and 15 min after perfusion of 10 μM NS5806 in the external solution. For recording of spontaneous excitatory post-synaptic currents (sEPSCs), same external and internal solutions for AP recording were used. Picrotoxin (0.1 mM) was added to the external solution to block inhibitory current. CA1 neuron were held at −70 mV for 2 min.

Previous studies used whole-cell patch clamp recordings at the soma of CA1 pyramidal cells to measure the whole-cell A-type K⁺ current (Chen, 2005; Good et al., 1996; Hall et al., 2015; Scala et al., 2015). To make comparisons to earlier studies, the same approach was employed. Briefly, whole-cell A-type K⁺ current (I_{A}) was elicited by depolarizing pulses to +40 mV from a holding potential of −100 mV in the presence of 20 mM tetraethylammonium (TEA) and 100 nM tetrodotoxin (TTX). In steady-state activation experiments, membrane potential was held at −100 mV, and I_{A} was evoked by a 200-ms depolarizing pulse from a first pulse potential of −80 mV to +80 my in 10-mV steps at 10-s intervals, Data were analyzed using the equation G_{K}=I_{K}/(Vₘ−Vᵣₑᵥ), where G_{K} is the membrane K⁺ conductance, Vₘ is the membrane potential, and Vᵣₑᵥ is the reversal potential for K⁺. To study steady-state inactivation of I_{A}, currents were elicited using 1-s conditioning pre-pulses from −110 mV to 0 mV before a 200-ms test pulse of +50 mV. After normalizing each current amplitude to the maximal current, amplitude obtained from the −110 mV pre-pulse was used as a function of the conditioning pre-pulse potential and fitted with the function I_{A}/I_{A-max}=1/(1+exp((V_{m1/2}−Vₘ)/k)), from which, an inactivation curve of I_{A} was obtained, and the V_{H} value (the voltage at which the current amplitude was half-inactivated) was calculated. The somatic whole-cell recordings provide information on the magnitude of somatic A-type K⁺ current, an important determinant of somatic excitability.

For HEK293 cell experiments, HEK293 cell lines were maintained as previously described (Jiang et al., 2004) and transiently transfected with cDNAs of Kv4.2 and KChIP4 together with cDNA encoding for GFP to identify cells successfully transfected. 12-16 h before recording, tetracycline was added to culture media to induce the expression of RyR2 WI or RyR2 E4872Q mutant. I_{A} was recorded with the same protocols as described above. Prior to I_{A} recording, the culture medium was replaced with a bath solution (NaCl, 125 mM; KCl, 2.5 mM; HEPES, 10 mM; MgCl₂, 1 mM; glucose, 10 mM; TEA, 20 mM; pH 7.4 adjusted with NaOH).

For recording the afterhyperpolarization current (I_{AHP}), brain slices were perfused with the carbogenated aCSF and pipettes were filled with I_{AHP} inner solution (KMeSO3, 130 mM; EGTA, 0.1 mM; HEPES, 10 mM; NaCl, 7 mM; MgCl2, 0.3 mM; di-tris-creatine, 5 mM; Tris-ATP, 2 mM and Na-GTP, 0.5 mM, pH 7.3 with KOH). I_{AHP} was evoked by a 100 ms depolarizing voltage step to +60 mV from a holding potential of −85 mV. Medium (I_{mAHP}) and slow (I_{sAHP}) amplitudes were measured at the peak of the current and 1 s after the end of the depolarizing pulse, respectively. All cells had a resting membrane potential more hyperpolarized than −60 mV, leak current smaller than 100 pA, and an input resistance of 150-350 Ω. Input resistance was determined from a −5 mV (100 ms) hyperpolarizing pulse applied at the beginning of each sweep. Access resistance was 80% electronically compensated and stable at <20 MΩ.

### In Vivo Two-Photon Ca²⁺ Imaging of CA1 Neurons

To determine whether limiting RyR2 open time can suppress AD-associated neuronal hyperactivity in vivo, double heterozygous 5×FAD^{+/-}/E4872Q^{+/-} mice were crossed with the heterozygous Thy-1 GCaMP6f^{+/-} transgenic mice (GP5.17, JAX 025393) to introduce the GCaMP6f^{+/-} transgene into each of the four genotypes (driven by the Thy1 promotor). In vivo two-photon imaging of GCaMP6f-expressing CA1 pyramidal neurons in each of the four genotypes to monitor spontaneous Ca²⁺ transients was performed.

Neuronal hyperactivity has been reported in anesthetized AD model mice in vivo using two-photon Ca^{2 +} imaging (Busche et al., 2008; Busche et al., 2012; Lerdkrai et al., 2018; Busche et al., 2019). To facilitate comparison, the same approach described originally by Busche et al. and used by others in the field (Busche et al., 2012; Busche et al., 2008; Delekate et al., 2014; Eichhoff and Garaschuk, 2011; Kim et al., 2016; Takano et al., 2007; Zott et al., 2019) was employed. Craniotomy was performed according to the protocol reported previously (Busche, 2018; Busche et al., 2012; Busche et al., 2008) with some modifications. Briefly, mice at different ages were anesthetized with 1-2% isoflurane (vol/vol in pure oxygen), and placed onto a heating plate (Homeothermic Monitor, Harvard Apparatus). The body temperature was monitored and controlled at 36.5-37.5° C. during the entire surgery and imaging procedure. After the removal of the skin, the skull was rinsed with artificial cerebral spinal fluid (aCSF: NaCl, 125 mM; KCl, 4.5 mM; NaH₂ PO₄, 1.25 mM; NaHCO₃, 26 mM; glucose, 20 mM; CaCl₂·2H₂O, 2 mM, and MgCl₂, 1 mM, pH to 7.3-7.4 with NaOH) and dried with cotton tips. A custom-made plastic recording chamber was glued to the skull with dental cement. The chamber was filled and kept perfusing with warm (37° C.) aCSF. The stereotactic coordinates of the hippocampus were located according to the mouse brain atlas and exposed. The craniotomy was filled with agarose (2-3%) and stabilized with a cover glass. Then, the animal was moved to the recording platform, and the isoflurane was gradually reduced to 0.5-0.8%. Besides the core body temperature, the respiratory and pulse rate were continuously monitored (MouseOx plus. STARR Life Science Corp.).

In vivo two-photon recordings were made using a custom-built two-photon microscope fed by a Ti:Sapph laser (Ultra II, ˜4W average power, 670-1080 nm, Coherent), using a water dipping Nikon objective lens (16x, NA 0.8) and Hamamatsu GaAsP PMT detectors. Image data were acquired using MATLAB, running on an open source scanning microscope control software named Scanimage (version 3.8.1, Howard Hughes Medical Institute/Janelia Farms, RRID:SCR_014307) (Pologruto et al., 2003). Imaging was performed at an excitation wavelength of 920 nm for GCaMP6f and fluorescence was captured using a 560 nm secondary dichroic and a 525-40 nm bandpass emission filter (Chroma Technologies). Time-series images were acquired at 15.63 Hz with a pixel density of 256×256 and a field of view size of ˜110 μm. For each view, spontaneous Ca²⁺ transients of hippocampal CA1 neurons were recorded for 5-10 min.

Image analyses were performed off-line using ImageJ (http://rsb.info.nih.gov/ij) and an open-source MATLAB program NeuroSeg (Guan et al., 2018). First, images were stabilized with ImageJ to reduce the x-y vibration, then regions of interest (ROI) were drawn around individual somata and the relative fluorescence change (ΔF/F) versus time traces for each ROI was generated using NeuroSeg. Ca²⁺ transients were identified as changes in ΔF/F that were three times larger than the standard deviation of the noise band. All recorded neurons were classified based on their activity rates as silent (0-0.2 transients/min), normal (0.2-20 transients/min), and hyperactive (≥20 transients/min) neurons following the definitions by Busche et al (Busche, 2018; Busche et al., 2012; Busche et al., 2008). Note that analyses of frequency distributions were performed using cells pooled from all animals, while analyses of mean frequency and fraction of silent, normal, and hyperactive cells were based on data from individual animals.

### Ex Vivo Two-Photon Ca²⁺ Imaging of CA1 Neurons

Ex vivo two-photon Ca²⁺ imaging was carried out as described previously with some modifications (Chen-Engerer et al., 2019). 5×FAD, RyR2 WT, and RyR2 mutant mice were cross-bred with the heterozygous Thy1-GCaMP6f transgenic mice (Chen et al., 2012; Chen et al., 2013) (GP5.17, JAX 025393) to express the GCaMP6f Ca²⁺ sensing probe (driven by the Thy1 promotor) in hippocampal neurons in each of the genotypes used. Transverse hippocampal slices (260 μm) were prepared as described above and kept in the carbogenated HEPES containing aCSF for at least 60 min before recording. Slices were then moved to a recording chamber containing carbogenated external solution (NaCl, 124 mM; KCl, 2.5 mM; NaH₂ PO₄, 1.25 mM; NaHCO₃, 24 mM; HEPES, 5 mM; glucose, 12.5 mM; MgCl₂, 2 mM; and CaCl₂, 2 mM; pH 7.4 adjusted with NaOH) and put under an up-right two-photon imaging system (SP8 DIVE, Leica, Germany) with CHAMELEON HEAD/PSU: ULTRA (H): 80 MHz (RoHS) laser (Coherent, UK). A 25× water-immersion objective with NA 0.95 (Leica, Germany) was used for imaging. Laser wavelength was set at 920 nm. Images were recorded with a resolution of 296×296 pixels at 16.77 fps. During recording, 0.5 μM tetrodoxin (TTX), 0.03 mM 2-amino-5-phosphonovaleric acid (APV), 0.02 mM 6,7-dinitroquinoxaline-2,3-dione (DNQX) and 0.1 mM picrotoxin were added to the external solution. Local drug application was performed by using a glass pipette with a resistance of −8 MO, which was connected to a modified pressurized perfusion system (ALA Scientific Instruments, Inc., USA). The pipette was filled with caffeine ringer solution (caffeine, 40 mM; CaCl₂, 2 mM; HEPES, 10 mM; KCl, 2.5 mM; MgCl₂, 1 mM; NaCl, 120 mM; NaH₂PO₄, 1.25 mM; pH 7.4 adjusted with NaOH). The pipette tip was placed at 15-20 μm from the soma of CA1 neuron. Caffeine (40 mM) was applied for 3 sec to induce Ca²⁺ release. The fluorescence intensity in each somatic ROI was corrected by background subtraction. A ROI immediately outside of the neuron was taken as background. Temporal fluorescence intensity changes in ROIs were expressed as relative changes in fluorescence intensity: ΔF/F=((F−F₀)/F₀). F₀ is defined as baseline fluorescence, which is the fluorescence intensity before a given stimulus, and F is the fluorescence recorded over time. ΔF/F values were calculated and plotted using NeuroSeg.

### Long-Term Potentiation Recording

Schaffer collateral fibers were stimulated at the CA3 subfield to record field excitatory postsynaptic potentials (fEPSPs) in the CA1 stratum radiatum of transverse hippocampal slices (300 pm) from all genotypes and drug-treated mice at different ages. After recovering at room temperature for 1 h (or 2 h for brain slices from drug-treated mice), hippocampal slices were allowed to recover in the recording chamber for additional 10 min. To evaluate basal synaptic transmission, different stimulation strengths (0 μA to 200 μA in steps of 20 μA) were applied and plotted fEPSP slopes versus the current input to compare the slope of input/output (I/O) curves of fEPSP. In the experiments that followed, stimulus current was adjusted so that fEPSP stabilized at 40-50% of maximum. Baseline was recorded for at least min until the differences among fEPSP slopes were within 10%. Long-term potentiation (LTP) was induced using a tetanic high-frequency stimulation (HFS; 4 trains of 100 pulses at 100 Hz, with 20-sec intervals). Synaptic responses were recorded for at least 60 min after tetanization and quantified as the slope of the evoked fEPSP as percentage of the baseline.

### Learning and Memory Tests

The learning and memory of mice with all genotypes were evaluated using the Morris water maze (MWM) test, the Barnes maze (BM) test, and/or the novel object recognition (NOR) test. Experiments were carried out blindly. For the MWM test, mice at different ages were trained to localize a hidden escape platform (10×10 cm) in a circular pool (116.84 cm in diameter, 50 cm in depth) (San Diego Instruments, CA) via distal visual cue. The platform was submerged 1-2 cm beneath the surface in water (22-24°C.), which was rendered opaque by addition of milk powder. The localization of the pool in relation to visual cues was maintained constantly during the entire task. The cues were distinct in color and shape. Digital division of the tracking area (pool) into four quadrants was performed by the SMART video tracking system, Smart 3.0 (Panlab Harvard Apparatus; Barcelona Spain). The escape platform was placed in the centre of the south-west quadrant for the entirety of the learning phase (4 training days) and digitally defined as target. Spatial training consisted of 4 days with 5 trials per mouse per day. Mice were released with their heads facing the pool wall at one of four entry locations (north, east, south and west) in a non-repetitive random order. Swimming was automatically video-tracked until the subject found the escape platform and remained on it (≥5 sec), or until a maximum of 60 seconds. Mice that did not locate the hidden platform within the time limit of 60 seconds were guided to the escape platform until they spent ≥10 seconds on it. In between trials (inter-trial interval ≥10 min), mice were housed in heated cages to avoid performance deficits due to exhaustion or hypothermia. The latency and swimming speed to reach the escape platform were recorded for comparison. After the learning phase, memory retention was evaluated by one probe trial 24 hours after the last training session. The escape platform was removed before mice were released from the north entry point into the pool. Their swimming was video-tracked for 60 seconds. The area at the location of the removed hidden platform was defined as the target and the south-west quadrant the target quadrant. The percentage of time mice spent in the target quadrant (including the target) were measured for comparison.

For the BM test, the size and characteristics of the device are as follows: a 92 cm diameter platform; the platform contains 20 holes, each 5 cm in diameter, equally distributed around the platform and separated by 7.5 cm; the device stands 105 cm above the floor. In one hole there is an escape box communicated with the platform through transparent plastic tunnels arranged in such a way that they cannot be seen from the platform. Similar to the MWM test the simultaneous use of a video-monitoring system is used to obtain automated behavioral recordings. Each trial lasts 3 min per mouse, with an inter-trial interval of 15 min, with four trials per day during the acquisition phase. The first phase (habituation), consisted of placing the mouse on the center of the platform and then, turning on the bright light as an aversive stimulus. Then the mouse was gently taken to the escape hole; once in the escape chamber, the light was turned off, and the mouse was kept inside for two additional minutes. During acquisition, mice were placed on the center of the platform and the light was turned on for 3 min, the latency to find the escape hole was recorded. If the mouse did not reach the escape hole within 3 min, the experimenter placed it at the entrance of the escape hole for 1 min, and then took it back to its home-cage. This protocol continued for 4 days. On day 5, 24 h after the last training day, the probe trial was conducted. The target hole was closed. The maze was rotated so that the target hole was closed and the maze was readjusted so that the holes were in the same position as during the training days. The mouse was then placed in the middle of the maze and allowed to explore the maze as before, The mouse was removed after 90 s. The probe trial was done in order to determine if the animal remembered where the target hole was located. The numbers of nose pokes to each hole were measured.

For the NOR test, mice were habituated for 10 min per mouse in an equally illuminated, odor-free, white, plastic box (40×40×50 cm³) embedded with fresh aspen shavings and shreds. In between each mouse trial the box was wiped with ethanol to avoid odor-induced stress. 24 hours after habituation, two identical objects ware placed at equal distance to each other and the corners of the box. Each mouse was placed into the center, and allowed to move freely for 10 min. Mice were video recorded during this familiarizing phase. Side preferences was evaluated by dividing the time a mouse spent exploring one object by the time they spent at the other object. Twenty-four hours later, one of the objects was replaced by a novel object. The other object remained constant. The selection of a familiar object to be replaced was random. Each mouse was again placed into the center of the box and allowed to move freely for another 10 min while videotaped. General exploration was evaluated by determining the time spent exploring the objects. The discrimination ratio describes the time a mouse explored the novel object divided by the total time it spent exploring (novel and familiar objects). The above experiments were carried out blindly.

### Biotinylation Assays

Biotinylation assays were performed according to the protocol described previously (Lin et al., 2010) with some modifications. Briefly, after 24-48 h of transfection with the Kv4.2 and KChIP4 cDNAs, transfected HEK293 cell lines expressing RyR2 WT or RyR2 E4872Q mutation were rinsed with ice-cold PBS for three times, surface proteins were biotinylated with 1.5 mg/ml sulfa-NHS-SS-biotin reagent (Pierce, Cat #PG82077) in PBS for 30 min on ice. Unbound biotin was quenched with ice-cold 50 mM glycine in PBS. Cells were lysed with ice-cold lysis buffer: 150 mM NaCl, 20 mM Tris-HCl, 1% NP40 and protease inhibitor cocktail (Roche, Cat #4693159001), sonicated and centrifuged at 12,000 g for 10 min. Cell lysates were incubated overnight at 4° C. with immobilized-Streptavidin agarose beads (Pierce, Cat #20349), unbound proteins were removed from the beads with 3 washes in lysis buffer. The bound proteins were eluted with 2×SDS sample buffer. Surface expressed proteins were separated by electrophoresis in 12% Tris-glycine SDS-PAGE and transferred to PVDF membranes. Western blots were probed with the following antibodies: rabbit anti-Kv4.2 (1:1000, abeam, Cat #ab 16719), rabbit anti-Rab4 (1:1000, Cell Signaling Technology, Cat #2167), goat anti-rabbit IgG secondary antibodies conjugated with horseradish peroxidase (1:10000, ThermoFisher, Cat #31460). The bound antibodies were detected using an enhanced chemiluminescence kit from Pierce.

### Immunoblotting

Immunoblotting analysis was carried out using the method described previously (Rosen et al., 2010).

### Immunohistochemical and Nissl Staining

Mice of different ages and genotypes were anesthetized and transcardially perfused with 10% neutral buffered formalin (NBF). Whole brains were removed and post-fixed in NBF for at least 24 h. The fixed brains were then embedded in paraffin after dehydration and diaphanization. For the IHC staining, paraffin-embedded brain tissue sections (5 μm) were immersed in xylene (5 min, 3 times), rehydrated in absolute ethanol (5 min, 3 times) followed by immersion in 95%, 80% and 70% solutions of ethanol (in water) (5 min each), Antigens were reactivated by treatment with 0.01 M citrate buffer (pH 6.0) for 2 min in microwave. Slides were washed in phosphate buffered saline (PBS: NaCl, 137 mM; KCl, 2.7 mM; Na₂HPO₄, 10 mM; KH₂PO₄, 2 mM, pH 7.4) and blocked with 10% normal horse serum in PBS for 10 min, then incubated with the primary antibody, the anti-β-amyloid peptide (total) antibody (Cell Signaling Technology, Cat #8243), for 12-16 h at 2-8° C. After washing with PBS, slides were incubated with biotinylated secondary antibody (Vector Laboratories, Cat #BA-1000) for 10 min, washed twice with PBS, and incubated with 3% H₂O₂ for 25 min for inactivation of endogenous peroxidase. Slides were then incubated with streptavidin-biotin-peroxidase for 30 min. Slides were covered with 3, 3′-diaminobenzidine (DAB) solution (0.06% in PBS containing 0.018% H₂O₂) for 1 to 5 min or until a brown precipitate could be observed. Identical conditions and reaction times were used for slides from different samples to allow comparison between immunoreactivity densities. Reaction was stopped by immersion of slides in distilled water. Counterstaining was performed with Harris hematoxilin. Coverslips were mounted with resinous mounting medium.

For the Nissl staining, paraffin-embedded brain tissue sections (5 μm) were immersed in xylene (5 min, 2 times), rehydrated in absolute ethanol (5 min, 2 times) followed by 95%, 75% and 50% solutions of ethanol in water (5 min each), then washed in distilled water for 2 times, 5 min each. Slides were stained in FD cresyl violet solution (FD Neurotechnologies, Baltimore, MD, USA) for 10 min, then, briefly rinsed in 100% ethanol and differentiated in 100% ethanol containing 0.1% glacia acetic acid for 1 min. Slides were then dehydrated in absolute ethanol (2 min, 4 times) followed by clearance in xylene (3 min, 2 times). Coverslip were mounted with resinous mounting medium.

### Single-Cell Ca²⁺ Imaging of HEK293 Cells

Intracellular cytosolic Ca²⁺ changes in stable, inducible HEK293 cells expressing RyR2 WT or RyR2 E4872Q mutant, transfected with presenilin 1 (PS1) WT, PS1 M146L, PS1 L286V or control plasmid (pcDNA3) were monitored using single-cell Ca²⁺ imaging and the fluorescent Ca²⁺ indicator dye Fura-2 AM, as described previously (Chen et al., 2014; Jiang et al., 2005; Jiang et al., 2004).

### Dendritic Spine Density Analysis

A FD Rapid GolgiStain kit (FD Neurotechnologies, Baltimore, MD, USA) was used for dendritic spine histological analysis by following the manufacturer's instructions as previously described (Zhao et al., 2015).

### Quantification and Statistical Analysis

All experiments were performed blindly to genotype, age and treatment. All data shown are medians and range (min and max), unless indicated otherwise. For small data sets (n number less than 15) or non-Gaussian distributed data, non-parametric methods were used. For large data sets and normally distributed data, parametric tests were performed. Wth respect to non-parametric analyses, for experiments with two groups, Mann-Whitney U test was used for unpaired samples. Wilcoxon matched-pairs signed rank test was used for paired samples. For experiments with 3 or more groups, Kruskal-Wallis test with Dunn-Bonferroni post hoc test and Friedman test with Dunn-Bonferroni post hoc test were used for independent samples or repeat measurements, respectively. Wth respect to parametric analyses, for experiments with two groups, Student's t test was used for unpaired samples. Paired t test was used for paired samples. For experiments with 3 or more groups, one-way ANOVA or two-way ANOVA test followed by Bonferroni post hoc test and repeated measure ANOVA test with Bonferroni post hoc test were used for independent samples or repeat measurements, respectively. P values smaller than 0.05 were considered statistically significant.

### REFERENCES

Alkon, D. L., Nelson, T. J., Zhao, W., and Cavaliar, S. (1998). Trends Neurosci 21, 529-537.
Bartsch, W., Spoiler, G., Strein, K., Muller-Beckmann, B., Kling, L., Bohm, E., Martin, U., and Borbe, H. O. (1990). European journal of clinical pharmacology 38 Suppl 2, S104-107.
Berridge, M. J. (2010). Pflugers Archiv : European journal of physiology 459, 441-449.
Bers, D. M. (2002). Nature 415, 198-205.
Bodhinathan, K., Kumar, A., and Foster, T. C. (2010). J Neurophysiol 104, 2586-2593.
Bogdanov, K. Y., Vinogradova, T. M., and Lakatta, E.G. (2001). Circ Res 88, 1254-1258.
Brager, D. H., and Johnston, D. (2007). J Neurosci 27, 13926-13937.
Bround, M. J., Asghari, P., Wamboit, R. B., Bohunek, L., Smits, C., Philit, M., Kieffer, T. J., Lakatta, E. G., Boheler, K. R., Moore, E. D., et al., (2012). Cardiovascular research 96, 372-380.
Brown, J. T., Chin, J., Leiser, S. C., Pangalos, M. N., and Randall A. D. (2011). Neurobiol Aging 32, 2109.e2101-2114.
Bruno, A. M., Huang, J. Y., Bennett, D. A., Marr, R. A., Hastings, M. L., and Stutzmann, G. E. (2012). Neurobiology of aging 33, 1001,e1001-1001.e1006.
Busche, M. A. (2018). Methods Mol Biol 1750, 341-351.
Busche, M. A., Chen, X., Henning, H. A., Reichwald, J., Staufenbiel, M., Sakmann, B., and Konnerth, A. (2012). Proc Nati Acad Sci USA 109, 8740-8745.
Busche, M. A., Eichhoff, G., Adelsberger, H., Abrarnowski, D., Wiederhold, K. H., Haass, C., Staufenbiel, M., Konnerth, A., and Garaschuk, O. (2008). Science (New York, NY) 321, 1686-1689.
Busche, M. A., and Konnerth, A. (2015). BioEssays news and reviews in molecular, cellular and developmental biology 37, 624-632,
Busche, M. A., and Konnerth, A. (2016). Philosophical transactions of the Royal Society of London Series B, Biological sciences 371.
Busche, M. A., Wegmann, S., Dujardin, S., Commins, C., Schiantarelli. J., Klickstein, N., Kamath, T. V., Carlson, C. A., Nelken, I., and Hyman, B. T. (2019). Nat Neurosci 22, 57-64.
Cacace, R., Heeman, B., Van Mossevelde, S., De Roeck, A., Hoogmartens, J., De Rijk, P., Gossye, H., De Vos, K., De Coster, W., Strazisar, M., et al., (2019). Acta neuropathologica 137, 901-918.
Chakroborty, S., Briggs, C., Miller, M. B., Goussakov, I., Schneider, C., Kim, J., Wicks, J., Richardson, J. C., Conklin, V., Carneransi, B. G., et al. (2012a). PloS one 7, e52056.
Chakroborty, S., Goussakov, I., Miller, M. B., and Stutzmann, G. E. (2009). The Journal of neuroscience: the official journal of the Society for Neuroscience 29, 9458-9470.
Chakroborty, S., Hill, E. S., Christian, D. T., Helfrich, R., Riley, S., Schneider, C., Kapecki, N., Mustaly-Kalimi, S., Seiler, F. A., Peterson, D. A., et al. (2019). Mol Neurodegener 14, 7.
Chakroborty, S., Kim, J., Schneider, C., Jacobson, C., Molgo, J., and Stutzmann, G. E. (2012b). J Neurosci 32, 8341-8353.
Chakroborty, S., and Stutzmann, G. E. (2014). European journal of pharmacology 739, 83-95.
Chan, C. H. (1990). Neurology 40, 1427-1432.
Chan, S. L., Mayne, M., Holden, C. P., Geiger, J. D., and Mattson, M. P. (2000). J Bial Chem 275, 18195-18200.
Chen-Engerer, H. J., Hartmann, J., Karl, R. M., Yang, J., Feske, S., and Konnerth, A. (2019). Nat Cornmun 10, 3223.
Chen, C. (2005). Biochem Biophys Res Commun 338, 1913-1919.
Chen, Q., Cichon, J., Wang, W., Qiu, L, Lee, S. J., Campbell, N. R., Destefino, N., Goard, M. J., Fu, Z., Yasuda, R., et al, (2012). Neuron 76, 297-308,
Chen, T. W., Wardill, T. J., Sun, Y., Pulver, S. R., Renninger, S. L., Baohan, A., Schreiter, E. R., Kerr, R. A., Orger, M. B., Jayaraman, V., et al. (2013). Nature 499, 295-300.
Chen, W., Wang, R., Chen, B., Zhong, X., Kong, H., Bai, Y. Zhou, Q., Xie, C., Zhang, J., Guo, A., et al, (2014). Nature medicine 20, 184-192.
Cirrito, J. R., Yamada, K. A., Finn, M. B., Sloviter, R. S., Bales, K. R., May, P. C., Schoepp, D. D., Paul, S. M., Mennerick, S., and Holtzman, D. M. (2005). Neuron 48, 913-922.
Coman, H., Nemes, Bogdan, (2017), International Journal of Gerentontology 11: 2-6.
Dana, H., Chen, T. W., Hu, A., Shields, B. C., Guo, C., Looger, L. L., Kim, D. S., and Svoboda, K. (2014). PloS one 9, e108897.
de Pins, B., Cifuentes-Diaz, C., Farah, A. T., López-Molina, L, Montalban, E., Sancho-Balsells, A., López, A., Ginés, S., Delgado-García, J. M., Alberch, J., et al, (2019). J Neurosci 39, 2441-2458.
Delekate, A., Füchtemeier, M., Schumacher, T., Ulbrich, C., Foddis, M., and Petzold, G .C. (2014). Nat Commun 5, 5422.
Demattos, R. B., Lu, J., Tang, Y., Racke, M. M., Delong, C. A., Tzaferis, J. A., Hole, J. T., Forster, B. M., McDonnell, P. C., Liu, F., et al. (2012). Neuron 76, 908-920.
Dickerson, B. C., Salat, D. H., Greve, D. N., Chua, E. F., Rand-Giovannetti, E., Rentz, D. M., Bertram, L., Mullin, K., Tanzi, R. E., Blacker, D., et al. (2005). Neurology 65, 404-411.
Eichhoff, G., and Garaschuk, O. (2011). Cold Spring Harbor protocols 2011, 1206-1216.
Furuichi, T., Furutama, D., Hakamata, Y., Nakai, J., Takeshima, H., and Mikoshiba, K. (1994). The Journal of neuroscience: the official journal of the Society for Neuroscience 14, 4794-4805.
Giannini, G., Conti, A., Mammarella, S., Scrobogna, M., and Sorrentino, V. (1995). J Cell Biol 128, 893-904.
Good, T. A., Smith, D. O., and Murphy, R. M. (1996). Biophys J 70, 296-304.
Guan, J., Li, J., Liang, S., Li, R., Li, X., Shi, X., Huang, C., Zhang, J., Pan, J., Jia, H., et al. (2018). Brain structure & function 223, 519-533.
Hall, A. M., Throesch, B. T., Buckingham, S. C., Markwardt, S. J., Peng, Y., Wang, Q., Hoffman, D. A., and Roberson, E. D. (2015). J Neurosci 35, 6221-6230.
Hardy, J., and Selkoe, D. J. (2002). Science 297, 353-356.
Hiess, F., Vallmitjana, A., Wang, R., Cheng, H., ter Keurs, H. E., Chen, J., Hove-Madsen, L., Benitez, R., and Chen, S. R. (2015). The Journal of biological chemistry 290, 20477-20487.
Hoffman, D. A., Magee, J. C., Colbert, C. M., and Johnston, D. (1997). Nature 387, 869-875.
Honig, L. S., Vellas, B., Woodward, M., Boada, M., Bullock, R., Borne, M., Hager, K., Andreasen, N., Scarpini, E., Liu-Seifert, H., et al. (2018). N Engl J Med 378, 321-330.
Jawhar, S., Trawicka, A., Jenneckens, C., Bayer, T. A., and Wirths, O. (2012). Neurobial Aging 33, 196.e129-140.
Jiang, D., Wang, R., Xiao, B., Kong, H., Hunt, D. J., Choi, P., Zhang, L., and Chen, S. R. W. (2005). Circ Res 97, 1173-1181.
Jiang, D., Xiao, B., Yang, D., Wang, R., Choi, P., Zhang, L., Cheng, H., and Chen, S. R. W. (2004). ProcNatlAcadSciUSA 101, 13062-13067.
Jung, S. C., and Hoffman, D. A. (2009). PLoS One 4, e6549,
Kamenetz, F., Tomita, T., Hsieh, H., Seabrook, G., Borchelt, D., Iwatsubo, T., Sisodia, S., and Malinow, R. (2003). Neuron 37, 925-937.
Karran, E., Mercken, M., and De Strooper, B. (2011). Nature reviewsDrua discovery 10, 698-712.
Kelliher, M., Fastborn, J., Cowburn, R. F., Bonkale, W., Ohm, T. G., Ravid, R., Sorrentino, V., and O'Neill, C. (1999). Neuroscience 92, 499-513,
Kennedy, M. E., Stamford, A. W., Chen, X., Cox, K., Cumming, J. N., Dockendort M. F., Egan, M., Ereshefsky, L., Hodgson, R. A., Hyde, L. A., et al. (2016). Science translational medicine 8, 363ra150.
Kerr, J. N., Greenberg, D., and Helmchen, F. (2005). Proc Natl Aced Sci USA 102, 14063-14068.
Kerrigan, T. L., Brown, J. T., and Randall, A. Q. (2014). Neuropharmacology 79, 515-524.
Keskin, A. D., Kekus; M., Adelsberaer, H., Neumann, U., Shimshek, D. R., Song, B., Zott, B., Peng, T., Forstl, H., Staufenbiel, M., et al, (2017). Proc Natl Aced Sci USA 114, 8631-8636.
Kim, D., Baik, S. H., Kang, S., Cho, S. W., Bee, J., Cha, M. Y., Sailor, M. J., Mook-Jung, I., and Ahn, K. H. (2016). ACS central science 2, 967-975.
Kim, H., Kim, B., Kim, H. S., and Cho, J. Y. (2020). Molecular brain 13, 17.
Kim, J., Jung, S. C., Clemens, A. M., Petralia, R. S., and Hoffman, D. A. (2007a). Neuron 54, 933-947.
Kim, J., Wei, D. S., and Hoffman, D. A. (2005). J Physiol 569, 41-57.
Kim, S., Yun, H. M., Baik, J. H., Chung, K. C., Nah, S. Y., and Rhim, H. (2007b). J Biol Chem 282, 32877-32889.
Ko, D. T., Hebert, P. R., Coffey, C. S., Curtis, J. P., Foody, J. M., Sedrakyan, A., and Krumholz, H. M. (2004). Arch Intern Med 164, 1389-1394.
Lacampagne, A., Liu, X., Reiken, S., Bussiere, R., Meli, A. C., Lauritzen, I., Teich, A. F., Zalk, R., Saint, N., Arancio, O., et al. (2017). Acta neuropathologica 134, 749-767.
Le Magueresse, C., and Cherubini, E. (2007). Hippocampus 17, 316-325.
Leão, R. N., Colom L. V., Borgius, L., Kiehn, O., and Fisahn, A. (2012). Neurobiol Aging 33, 2046-2061.
Leinert H (1987). U.S. Pat. No. 4,697,022.
Lerdkrai, C., Asavapanumas, N., Brawek, B., Kovalchuk, Y., Mojtahedi, N., Olmedillas Del Moral, M., and Garaschuk, O. (2018). Proc Natl Acad Sci USA 115, E1279-e1288.
Lin, L., Sun, W., Wkenheiser, A. M., Kung, F., and Hoffman, D. A. (2010). Mol Cell Neurosci 43, 315-325.
Liu, J., Supnet, C., Sun, S., Zhang, H., Good, L., Popugaeva, E., and Bezprozvanny, I. (2014). The role of ryanodine receptor type 3 in a mouse model of Alzheimer disease. Channels (Austin, Tex) 8, 230-242.
Loera-Valenica et al. (2019), J. Intern Med. 286:398-437.
Magee, J., Hoffman, D., Colbert, C., and Johnston, D. (1998). Annu Rev Physiol 60, 327-346.
Malig, T., Xiao, Z., Chen, S. R. W., and Back, T. G. (2016). Bioorganic & Medicinal Chemistry Letters 26, 149-153.
Mandikian, D., Bocksteins, E., Parajuli, L. K., Bishop, H. I., Cerda, O, Shigemoto, R., and Trimmer, J. S., (2014). J Comp Neural 522, 3555-3574.
Maxwell, J. T., Domeier, T. L., and Blatter, L. A. (2012). American journal of physiologyHeart and circulatory physiology 302, H953-963.
Morohashi, Y., Hatano, N., Ohya, S., Takikawa, R., Watabiki, T., Takasugi, N., Imaizumi, Y., Tomita, T., and Iwatsubo, T. (2002). J Biol Chem 277, 14965-14975.
Murayama, T., and Ogawa, Y (1996). J Biol Chem 271, 5079-5084.
Nelson, M. T., Cheng, H., Rubart, M., Santana, L. F., Barley, A. D., Knot, H. J., and Lederer, W. J. (1995). Science (New York, NY) 270, 633-637.
Nichols, A. J., Sulpizio, A. C., Ashton, D. J., Hieble, J. P., and Ruffolo, R. R., Jr, (1989). Chirality 1, 265-270.
Noh, W., Pak, S., Choi, G., Yang, S., and Yang, S. (2019). Frontiers in cellular neuroscience 13, 265.
Nuriel, T., Angulo, S. L. Khan, U., Ashok, A., Chen, Q., Figueroa. H. Y., Emrani, S., Liu, L., Herman, M Barrett, G., et al (2017). Nat Commun 8, 1464.
O'Brien, J. L., O'Keefe, K. M., LaViolette, P. S., DeLuca, A. N., Blacker, D., Dickerson. B. C., and Sperling, R. A. (2010). Neurology 74, 1969-1976.
Oakley, H., Cole, S. L., Logan, S., Maus, E., Shao, P., Craft, J., Guillozet-Bongaarts, A., Ohno, M., Disterhoft, J., Van Eldik, L., et al. (2006). The Journal of neuroscience: the official journal of the Society for Neuroscience 26, 10129-10140.
Oda, T., Yang, Y., Uchinourni, H., Thomas, D. D., Chen-Izu, Y., Kato, T., Yamamoto, T., Yano, M., Cornea, R. L., and Bers, D. M. (2015). J Mol Cell Cardiol 85, 240-248,
Oo, Y. W., Gomez-Hurtado, N., Walweel, K., van Heiden, D. F., Imtiaz, M. S., Knollmann, B. C., and Laver, D. R. (2015). Essential Role of Calmodulin in RyR Inhibition by Dantrolene. Mol Pharmacol 88, 57-63.
Oules, B., Del Prete, D., Greco, B., Zhang, X., Lauritzen, I., Sevalle, J., Moreno, S., Paterlini-Brechot, P., Trebak, M., Checler, F., et al, (2012). The Journal of neuroscience: the official journal of the Society for Neuroscience 32, 11820-11834.
Packer, M., Bristow, M. R., Cohn, J. N., Colucci, W. S., Fowler, M. B., Gilbert, E. M., and Shusterman, N. H. (1996). The New England journal of medicine 334, 1349-1355.
Peng, J., Liang, G., Inan, S., Wu, Z., Joseph, D. J., Meng, Q., Peng, Y., Eckenhoff, M. F., and Wei, H. (2012). Neuroscience letters 516, 274-279.
Peron, S., Chen, T. W., and Svoboda, K. (2015). Current opinion in neurobiology 32, 115-123.
Pologruto, T. A., Sabatini, B. L., and Svoboda, K. (2003). Biomedical engineering online 2, 13.
Priori, S. G., and Chen, S. R. (2011). Circulation research 108, 871-883.
Rhodes, K. J., Carroll, K. I., Sung, M. A., Doliveira, L. C., Monaghan, M. M., Burke, S. L., Strassle, B. W., Buchwalder, L., Menegola, M., Cao, J., et al, (2004). J Neurosci 24, 7903-7915.
Risher, W. C., Ustunkaya, T., Singh Alvarado, J., and Eroglu, C. (2014). PLoS One 9, e107591.
Rosen, R. F., Tomidokoro, Y., Ghiso, J. A., and Walker, L. C. (2010). SDS-PAGE/immunoblot detection of Abeta multimers in human cortical tissue homogenates using antigen-epitope retrieval. J Vis Exp.
Rybalchenko, V., Hwang, S. Y., Rybalchenko, N., and Koulen, P. (2008). Int J Biochem Cell Biol 40, 84-97.
Sandler, V. M., and Barbara, J. G. (1999). J Neurosci 19, 4325-4336.
SanMartin, C. D., Veloso, P., Adasme, T., Lobos, P., Bruna, B., Galaz, J., Garcia, A., Hartel, S., Hidalgo, C., and Paula-Lima, A. C. (2017). Front Mol Neurosci 10, 115.
Sato, T. R., Gray, N. W., Mainen, Z. F., and Svoboda, K. (2007). PLoS Biol 5, e189.
Scala, F., Fusco, S., Ripoii, C., Piacentini, R., Li Puma, ED., Spinelli, M., Laezza, F., Grassi, C., and D'Ascenzo, M. (2015). Neurobiol Aging 36, 886-900.
Serodio, P., and Rudy, B. (1998). J Neurophysiol 79, 1081-1091.
Sevigny, J., Chiao; P., Bussiere, T., Weinreb, P. H., Williams, L., Maier, M., Dunstan, R., Salloway, S., Chen, T., Ling, Y., et al. (2016). Nature 537, 50-56.
Shiratsuchi M, et al (1987). Chem Pharm Bull (Tokyo) 35(9): 3691-3698,
Šišková, Z., Justus, D., Kaneko, H., Friedrichs, D., Henneberg, N., Beutel, T., Pitsch, J., Schoch, S., Becker, A., von der Kammer, H., et al. (2014), Neuron 84, 1023-1033.
Smith, I. F., Hitt, B., Green, K. N., Oddo, S., and LaFeria, F. M. (2005). Journal of neurochemistry 94, 1711-1718.
Stargardt, A., Swaab, D. F., and Bossers, K. (2015). Neurobiol Aging 36, 1-11.
Stoschitzky, K., Koshucharova, G., Lercher, P., Maier, R., Sakotnik, A., Klein, W., Liebmann, P. M., and Lindner, W. (2001). Chirality 13, 342-346.
Sun, W., Maffie, J. K., Lin, L., Petralia, R. S., Rudy, B., and Hoffman, D. A. (2011). Neuron 71, 1102-1115.
Takano, T., Han, X., Deane, R., Zlokovic, B., and Nedergaard, M. (2007). Ann N Y Aced Sci 1097, 40-50.
Takeshima, H., Komazaki, S., Hirose, K., Nishi, M., Noda, T., and lino, M. (1998). Embo J 17, 3309-3316.
Tamagnini, F., Novelia, J., Kerrigan, T. L., Brown, J. T., Tsaneva-Atanasova, K., and Randall, A. D. (2015). Frontiers in cellular neuroscience 9, 372.
Ting, J. T., Daigle, T. L., Chen, Q., and Feng, G. (2014). Methods in molecular biology (Clifton, NJ) 1183, 221-242.
Ting, J. T., Lee, B. R., Chong, P., Soler-Llavina, G., Cobbs, C., Koch, C., Zeng, H., and Lein, E. (2018). J Vis Exp.
Utili, R., Boitnott, J. K., and Zimmerman, H. J. (1977). Gastroenterology 72, 610-616.
van de Vrede, Y., Fossier, P., Baux, G., Joels, M., and Chameau, P, (2007). Pflugers Arch 455, 297-308.
van Zwieten, P. A.(1993). Cardiology 82 Supp13, 19-23.
Varga, A. W., Yuan, L. L., Anderson, A. E., Schrader, L. A., Wu, G. Y., Gatchel, J. R., Johnston, D., and Sweatt, J. D. (2004). J Neurosci 24, 3643-3654.
Wang, J., Ono, K., Dickstein, D. L., Arrieta-Cruz, I., Zhao, W., Qian, X., Lamparello, A., Subnani, R., Ferruzzi, M., Pavlides, C., Ho, L., Hof, P. R., Teplow, D. B. and Pasinetti, G. M. (2011). Neurobiol Aging, 32(12): 2321.e1-2321.e12.
Waring, J. F., Anderson, D. J., Kroeger, P. E., Li, J., Chen, S. F., Hooker, B. A., Gopalakrishnan, M., and Briggs, C. A. (2012). Journal of Drug Metabolism & Toxicology 3, 1000115.
Weller, J and Budson, A. (2018), F1000 Research: 7 (F1000 Faculty Rev): 1161.
Wu, B., Yamaguchi, H., Lai, F. A., and Shen, J. (2013). Proceedings of the National Academy of Sciences of the United States of America 110, 15091-15096.
Wu, Z., Yang, B., Liu, C., Liang, G., Eckenhoff, M. F., Liu, W., Pickup, S., Meng, Q., Tian, Y., Li, S., et al. (2015). Alzheimer disease and associated disorders 29, 184-191.
Xiang, H., Kovacs, I., and Zhang, Z. (2004). Brain Res Mol Brain Res 128, 103-111.
Xu, J., Kang, N., Jiang, L., Nedergaard, M., and Kang, J. (2005). J Neurosci 25, 1750-1760.
Yamamoto, K., Tanei, Z., Hashimoto, T., Wakabayashi, T., Okuno, H., Naka, Y., Yizhar, O., Fenno, L. E., Fukayama, M., Bito, H., et al. (2015), Cell Rep 11, 859-865.
Yang, E. J., Mahmood, U., Kim, H., Choi, M., Choi, Y., Lee, J. P., Cho, J. Y., Hyun, J. W., Kim, Y. S., Chang, M. J., et al. (2018). Free Radic Bial Med 126, 221-234.
Zhang, H., Sun, S., Herreman, A., De Strooper, B., and Bezprozvanny, I. (2010). J Neurosci 8566-8580.
Zhang, J., Zhou, Q., Smith, C. D., Chen, H., Tan, Z., Chen, B., Nani, A., Wu, G., Song, L. S., Fill, M., et al. (2015). The Biochemical journal 470, 233-242.
Zhao, F., Li, P., Chen, S. R., Louis, C. F., and Fruen, B. R. (2001). Molecular mechanism and isoform selectivity. J Biol Chem 276, 13810-13816.
Zhao, Q. R., J. M., Yao, J. J., Zhang, Z. Y., Ling, C., and Mei, Y. A. (2015). Scientific reports 5, 11768.
Zhao, X., Weisleder, N., Han, X., Pan, Z., Farness, J., Brotto, M., and Ma, J. (2006). The Journal of biological chemistry 281, 33477-33486.
Zhou, Q., Xiao, J., Jiang, D., Wang, R., Vembaiyan, K., Wang, A., Smith, C.D., Xie, C., Chen, W., Zhang, J., et al, (2011). Nature medicine 17, 1003-1009,
Zott, B., Simon, M. M., Hong, W., Unger, F., Chen-Engerer, H. J., Frosch, M. P., Sakmann, B., Walsh, D. M., and Konnerth, A. (2019). Science 365, 559-565.

1. A method of treating or preventing at least one of the following in a subject in need thereof (a) memory loss; (b) long-term potentiation impairment; (c) neuronal cell death; and (d) neuronal hyperactivity, comprising administering a therapeutically effective amount of R-carvedilol, a metabolite of R-carvedilol, or a salt thereof to the subject.

2. The method of claim 1, wherein said method is for treating or preventing Alzheimer's Disease.

3. The method of claim 2, wherein the subject has been diagnosed as having Alzheimer's Disease.

4. The method of claim 2, wherein the subject is at risk of developing Alzheimer's Disease.

5. The method of claim 1, wherein said method treats or prevents cognitive decline.

6. The method of claim 1, wherein said method at least partially restores cognitive function.

7. The method of claim 1, wherein said method treats or prevents memory loss in the subject.

8. The method of claim 2, wherein said Alzheimer's Disease is preclinical Alzheimer's Disease, Mild cognitive impairment, or Alzheimer's dementia.

9. The method of claim 8, wherein said Alzheimer's Disease is Alzheimer's dementia.

10. The method of claim 1, wherein the R-carvedilol, metabolite of R-carvedilol, or salt thereof is administered parenterally.

11. The method of claim 10, wherein the R-carvedilol, metabolite of R-carvedilol, or salt thereof is administered intravenously, subcutaneously, or intramuscularly.

12. The method of claim 1, wherein the R-carvedilol, metabolite of R-carvedilol, or salt thereof is administered orally.

13. The method of claim 1, wherein the R-carvedilol, metabolite of R-carvedilol, or salt thereof is administered intranasally.

14. The method of claim 1, wherein the R-carvedilol, metabolite of R-carvedilol, or salt thereof is administered to cerebral spinal fluid (CSF).

15. The method of claim 1, wherein the therapeutically effective amount is from about 1.6 mg to about 50 mg daily.

16. The method of claim 1, wherein the therapeutically effective amount is from about 4 mg to about 32 mg twice a day.

17. The method of claim 1, wherein the therapeutically effective amount is about 12.5 mg daily.

18. The method of claim 1, wherein the therapeutically effective amount is about 8 mg twice a day.

19. The method of claim 1, wherein the therapeutically effective amount is from about 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, or 16 and up to about 65, 60, 55, 50, 45, 40, 35, 30, 25, or 20 mg daily.

20. A pharmaceutical composition for use in the treatment or prevention of at least one of the following (a) memory loss; (b) long-term potentiation impairment; (c) neuronal cell death; and (d) neuronal hyperactivity in a subject in need thereof comprising a pharmaceutically active ingredient consisting of R-carvedilol, a metabolite of R-carvedilol, or salt thereof, together with a pharmaceutically acceptable carrier.

21. The pharmaceutical composition of claim 20, wherein the pharmaceutical composition is for use in treating or preventing Alzheimer's Disease.

22. The pharmaceutical composition of claim 20, wherein the pharmaceutical composition is formulated as a nasal spray, aerosol or nasal drop.

23. The pharmaceutical composition of claim 20, wherein the pharmaceutical composition is formulated for oral administration.

24. The pharmaceutical composition of claim 20, wherein the pharmaceutical composition is formulated for parenteral administration.

25. The pharmaceutical composition of claim 24, wherein the pharmaceutical composition is formulated for subcutaneous, intramuscular, or intravenous administration.

26. (canceled)

27. (canceled)

28. (canceled)

29. (canceled)
